(19) United States
(12) Patent Application Publication
(10) Pub. No.: US 2026/0166046 A1
(43) Pub. Date: Jun. 18, 2026

(54) Inhibitors of methionine aminopeptidase-2 and methods of preparation and uses thereof

(71) Applicant: Primetime Life Sciences LLC, Germantown, MD (US)

(72) Inventor: Janak K Padia, Germantown, MD (US)

(73) Assignee: Janak K Padia, Germantown, MD (US)

(21) Appl. No.: 19/103,290

(22) PCT Filed: Aug. 14, 2023
(86) PCT No.: PCT/US2023/072133

§ 371 Date: Feb. 12, 2025

Related U.S. Application Data
(60) Provisional application No. 63/398,606, filed on Aug. 17, 2022.

(51) Int. Cl. A61K 31/5377

(52) U.S. Cl. CPC A61K 31/5377

(57) ABSTRACT

This invention pertains to the discovery and development of novel inhibitors for Methionine Aminopeptidase 2 (MetAP2). The MetAP2 inhibitors have demonstrated efficacy in therapeutic applications for treating various conditions, including parasitic diseases, cancer, obesity, inflammation, arthritis, sickle cell disease, and autoimmunity, among others. The scope of the invention extends beyond the inhibitors themselves to incorporate methods for their synthesis or use, kits incorporating these inhibitors, devices or formulations that include these inhibitors, and related components

### CROSS REFERENCE TO RELATED APPLICATION

This application claims priority to U.S. Provisional Application No. 63/398,606, filed on Aug. 17, 2022, the disclosure of which is incorporated by reference herein.

### STATEMENT REGARDING FEDERALLY SPONSORED RESEARCH AND DEVELOPMENT

This invention was made with government support under R44A1165220, awarded by the National Institutes of Health. The government has certain rights in the invention.

### FIELD OF THE INVENTION

This invention pertains to the discovery and development of novel inhibitors for Methionine Aminopeptidase 2 (MetAP2). The MetAP2 inhibitors have demonstrated efficacy in therapeutic applications for treating various conditions, including parasitic diseases, cancer, obesity, inflammation, arthritis, sickle cell disease, and autoimmunity, among others. The scope of the invention extends beyond the inhibitors themselves to incorporate methods for their synthesis or use, kits incorporating these inhibitors, devices or formulations that include these inhibitors, and related components

### BACKGROUND OF THE INVENTION

Methionine aminopeptidase 2 (MetAP2) is a member of the dimetallohydrolase family. MetAP2 is found in all organisms and is especially important because of its critical role in tissue repair and protein degradation. It has been exploited as a drug target (review (Grocin et al., Trends Pharmacol Sci, 2021,870-882)) by academic and federal investigators and also by pharmaceutical companies for the treatment of parasitic disease (Arico-Muendel et al., Bioorg Med Chem Lett, 2009,5128-5131; Arico-Muendel et al., J Med Chem, 2009,8047-8056; Chen et al., Chemistry & biology, 2009,193-202; Galkin et al., J Biol Chem, 2014,10502-10509; Han & Weiss, Expert Opinion on Therapeutic Targets, 2018,903-915; Killough et al., Science, 1952,71-72; Kulakova et al., Antimicrobial agents and chemotherapy, 2014,7303-7311; Laupland & Church, BMC Infect Dis, 2005,72; Osnat Herzberg, U.S. Ser. No. 16/551,628,2019; Padia et al., Antimicrobial agents and chemotherapy, 2020,e00582-00520; Zheng et al., 2015), cancer(Ingber et al., Nature, 1990,555-557; Kusaka et al., Biochemical and Biophysical Research Communications, 1991,1070-1076), obesity(Burkey et al., Journal of Pharmacology and Experimental Therapeutics, 2018,301-313), inflammation(Zampino et al., Innovation in Aging, 2020,126-127), rheumatoid arthritis(Lazarus et al., Inflammation research, 2008,18-27), sickle cell disease(Demers et al., Blood advances, 2021,1388-1402) and autoimmunity(Priest et al., Clinical & Experimental Immunology, 2009,514-522).

Fumagillin was discovered as an inhibitor of MetAP2 (Sin et al., Proc Natl Acad Sci USA, 1997,6099-6103). Fumagillin is a water-insoluble antibiotic derived from Aspergillus fumigatus; it was discovered in 1949 and originally used in humans as an amebicide. Topical fumagillin has been used to treat microsporidial keratoconjunctivitis caused by Encephalitozoon hellem, Encephalitozoon cuniculi, Encephalitozoon (Septata) intestinalis, and, with less success, Vittaforma corneae (Nosema corneum) in AIDS patients (Diesenhouse et al., American journal of ophthalmology, 1993,293-298). Oral fumagillin has been used successfully for Encephalitozoon beineusi infections inherently resistant to albendazole. (Champion et al., American journal of transplantation: official journal of the American Society of Transplantation and the American Society of Transplant Surgeons, 2010,1925-1930).

Fumagillin is a beneficial compound in human medicine and apiculture, but undesirable side effects are reported.(Conteas et al., The American journal of tropical medicine and hygiene, 2000,121-127; Didier, Antimicrobial agents and chemotherapy, 1997,1541-1546; Didier et al., Antimicrobial agents and chemotherapy, 2006,2146-2155; Ingber et al., Nature, 1990,555-557; Lanternier et al., Transpl Infect Dis, 2009,83-88; Molina et al., Aids, 2000,1341-1348; Molina et al., The New England journal of medicine, 2002,1963-1969; Yanase et al., Cancer Res, 1993,2566-2570) Repeated administration for an extended period of time of fumagillin caused severe body weight loss of >15% in human test subjects.(Yanase et al., Cancer Res, 1993,2566-2570) In 1952, it was reported that fumagillin was essentially nontoxic to humans at oral doses of up to 50 mg daily for 2 weeks to treat intestinal amebiasis (Killough et al., Science, 1952,71-72). However, no weight loss was observed in test subjects. In a more recent study, significant bone marrow toxicity of fumagillin was reported with 4 patients of a group of 11 patients at the highest dosage administered (60 mg). These effects ceased within days of the treatment being terminated and administered fumagillin orally up to 60 mg daily for 2 weeks to treat microsporidiosis in patients with HIV infection (Lanternier et al., Transpl Infect Dis, 2009,83-88). The EU has approved fumagillin as an orphan antimicrosporidiosis drug for treating immunocompromised patients. It is also effective when used topically in treating microsporidial keratoconjunctivitis and is recommended by the CDC for refractive microsporidial eye infections. In contrast to the amebiasis patients, who exhibited no adverse side effects during fumagillin treatment, 33% of the immune-compromised patients treated with fumagillin had a reversible toxic effect on bone marrow (primarily Thrombocytopenia), with spontaneous platelet count recovery within 1-2 weeks after halting therapy.

A fumagillin analog (Compound 9) has shown in vitro MetAP2 inhibition, antigiardiasis and antiamebiasis activities, and in vivo antigiardiasis activity in mouse models(Padia et al., Antimicrobial agents and chemotherapy, 2020,e00582-00520).

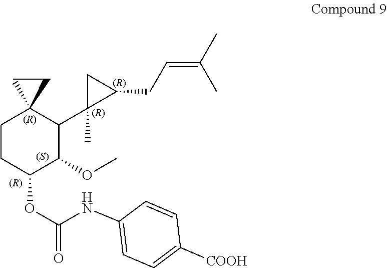

The therapeutic use of fumagillin has been hampered by its non-drug-like profiles, such as oral bioavailability, aqueous solubility, and solubility. Fumagillin is an unstable compound, exhibiting light, temperature, humidity, and pH-dependent degradation(Agner et al., Acta Pharm Hung, 2003,41-45). Moreover, cellular esterases can hydrolyze the C6 ester bond once the compound is transported into the cell. While drugs can be easily protected from light using formulation in non-translucent gel capsules, the temperature sensitivity reduces shelf life. It requires refrigeration, a disadvantage that limits broad use in the clinic. The sensitivity to low pH, such as present in the stomach, reduces the effective drug concentration by the time it reaches the intestine. This necessitates higher dosing, which in turn increases the potential of toxic effects.

There were many analogs designed and developed to improve drug-like properties, for example, TNP-470(Yanase et al., Cancer Res, 1993,2566-2570), beloranib (Hughes et al., Obesity, 2013,1782-1788), ZNG-1061 (Wentworth & Colman, Diabetes Obes Metab, 2020,1215-1219), and ZNG-1258 (Pottorf et al., JCI Insight, 2020). Although these compounds had better profiles in vitro and in vivo studies, they failed in clinical trials. Therefore, there is a need for a fumagillin analog with better drug-like properties (for example, potency, stability, solubility, efficacy, and safety profiles). In addition, there exists an ongoing and unmet need for therapeutic agents that are more potent, more stable, and less toxic than fumagillin.

Boron has unique characteristics and chemical properties. It has special properties and useful medicinal chemistry related to the design of new drugs. Boron belongs to the same period of the periodic table as carbon and nitrogen, two essential atoms that form the backbone of life; boron has the potential to play an important role in drug design. Nevertheless, medicinal chemists have not explored it to its full potential in drug design. (Baker et al., Future medicinal chemistry, 2009,1275-1288) (Nocentini et al., Expert opinion on therapeutic patents, 2018,493-504) Boron is a strong Lewis acid, has an empty p-orbital, and is electrophilic. Boron can form a dative bond (coordinate covalent bond) with biological nucleophiles, such as hydroxyl and amine groups present in enzyme residues, carbohydrates, and nucleic acids, since it has an empty p-orbital; many of the biological activities of the boron-containing compounds has been attributed to this characteristic of the boron atom. In addition, the boron center can be easily converted from neutral trigonal planar sp2 to tetrahedral sp3 hybridization under certain physiological conditions. (Ban & Nakamura, The Chemical Record, 2015,616-635; Yang et al., MedChemComm, 2018,201-211). We envisioned incorporating boron in the fumagillin analog at the C6 portion can modulate pharmacodynamics and pharmacokinetic properties to improve stability, efficacy, and safety profiles.

The first boron-containing drug on the market is bortezomib (Velcade®), a dipeptide boronic acid for treating multiple myeloma (the first-in-class proteasome inhibitor).(Richardson et al., Cancer control, 2003,361-369) Other drugs approved by FDA include tavaborole (Kerydin®) for the treatment of onychomycosis (Markinson et al., J Am Podiatr Med Assoc, 2018,12-19)and crisaborole (Eucrisa®) for the treatment of mild to moderate atopic dermatitis (Freund et al., FEBS letters, 2012,3410-3414; Nazarian & Weinberg, Curr Opin Investig Drugs, 2009,1236-1242) [5-7]. Several boron-containing compounds are currently in clinical phase studies, being investigated for different therapeutic applications, including psoriasis, human African trypanosomiasis (sleeping sickness), and hepatitis C (Chong et al., J Med Chem, 2019,3254-3267; Jacobs et al., PLoS Negl Trop Dis, 2011,e1151; Nocentini et al., Expert opinion on therapeutic patents, 2018,493-504).

Drug molecules can permeate intestinal membranes via paracellular and transcellular (Sundqvist et al., CPT: pharmacometrics & systems pharmacology, 2015,243-254) routes. However, the high selectivity of biological membranes prohibits a set of potential drug candidates that can be passively transported with certain physiological parameters. Active uptake via molecular transporters allows some molecules to circumvent cell membranes (Martinez & Amidon, The Journal of Clinical Pharmacology, 2002,620-643). Many promising drug candidates with high potency and selectivity in vitro for the desired target are poor substrates for these active transport processes. Because of these limitations, they have very limited or no cell membrane permeability, and hence they have poor oral bioavailability to be active in vivo.

Nevertheless, the drug strategy has improved intestinal membrane permeability for many drugs (Sundqvist et al., CPT: pharmacometrics & systems pharmacology, 2015,243-254). Typical drug designs using the non-specific approach of covalently attaching appropriate hydrophilic or lipophilic moieties to the molecule of interest to manage solubility and passive permeability have had some success. Over the last 20 years, more rationalized drug strategies have emerged in which moieties are covalently attached to the molecule of interest to selectively target certain membrane transporters and enzymes. These covalent drug strategies offer tremendous potential for modulating drug bioavailability and selectivity. Thus, there is a need to incorporate important parameters when applying this approach, e.g., synergetic and/or additive effects on efficacy, distribution, metabolism, excretion, and toxicity. It requires fine-tuned aqueous solubility, pKa, hydrophilicity, lipophilicity, clogP, and enzymatic or non-enzymatic cleavage rate.

### SUMMARY OF THE INVENTION

The present invention relates to compounds that are inhibitors of methionine aminopeptidase 2 (MetAP2), to process of preparing these compounds, their salts, prodrugs, and metabolites, pharmaceutical compositions containing these compounds, and to methods of using these compounds for treating a wide variety of medical conditions, diseases or disorders. In one aspect, the invention provides compounds having the structure of Formula I, including pharmaceutically acceptable prodrugs, metabolites, and isomers thereof

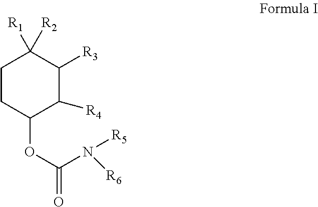

wherein
R₁ is selected from a group of —CH₂Cl, and —CH₂Br;
R₂ is selected from a group of hydrogen and hydroxyl;
R₁ and R₂ together can form

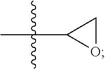 [SMILES: *C1CO1]

R₃ is selected from a group of hydrogen and substituted or unsubstituted alkyl;
R₄ is selected from a group of hydrogen and alkoxy;
R₅ is selected from a group of hydrogen and alkyl; and
R₆ is selected from a group of substituted or unsubstituted alkyl boronic acids, substituted or unsubstituted alkyl boronic acid esters, substituted or unsubstituted aryl boronic acids, substituted or unsubstituted aryl boronic acid esters, substituted or unsubstituted heterocyclic boronic acids, substituted or unsubstituted heterocyclic boronic acid esters;
NR₅R₆ can form a substituted or unsubstituted heterocyclic boronic acids,
substituted or unsubstituted heterocyclic boronic acid esters.

According to one embodiment of the invention a pharmaceutical composition comprising a compound of Formula I or a pharmaceutically acceptable salt, ester, or prodrug or metabolite thereof in association with a pharmaceutically acceptable diluent or carrier.

According to one embodiment of the invention a pharmaceutical composition comprising a compound of Formula I or a pharmaceutically acceptable salt, ester or prodrug, or metabolite thereof in association with a pharmaceutically acceptable diluent or carrier to form a formulation system for delivering the compound.

According to one embodiment of the invention a pharmaceutical composition comprising a compound of Formula I or a pharmaceutically acceptable salt, ester, or prodrug or metabolite thereof in a combination of other pharmaceutically active agent(s).

According to one embodiment of the invention a compound of Formula I or a pharmaceutically acceptable salt, ester or prodrug, or metabolite thereof for use in therapy.

According to one embodiment of the invention the use of a compound of Formula I or a pharmaceutically acceptable salt, ester or prodrug or metabolite thereof in the preparation of a medicament for the treatment and or prevention of parasitic disease, cancer, obesity, angiogenesis, inflammation, and immune disorders.

According to one embodiment of the invention a process of producing a compound of Formula I or its pharmaceutically acceptable salt or prodrug or metabolite.

According to another aspect, a method for treating or preventing a disease or condition by inhibiting MetAP2 is provided. It includes the step of administering a compound as provided herein. Any of the methods or uses provided herein may include administering to a subject a therapeutically effective amount of a compound as provided herein, including salt or polymorph thereof, or a pharmaceutical composition that includes such compounds.

The invention relates to formulation systems loaded with compounds of this invention mentioned above which said systems have improved biopharmaceutical properties for solubility, drug concentration in the target tissue (s), in vivo efficacy and safety, improved quality (fineness and homogeneity of the particles, drug inclusion) and improved physical stability of the particulate formulation (no aggregation or gel formation). The compound of this invention can be appropriately formulated for desired delivery systems such as oral drug delivery (immediate release, delayed-release, prolonged-release, modified release), parenteral drug delivery, ophthalmic drug delivery, nasal drug delivery, rectal drug delivery, intestinal-specific delivery, colon-specific drug delivery, topical drug delivery, and CNS or brain drug delivery by powder injection; or by buccal, sublingual, or intranasal absorption. Pharmaceutical compositions may be formulated in unit dose form or multiple or subunit doses.

The manner in which the compounds or their pharmaceutical composition set forth herein may be administered can vary. According to one embodiment of the invention the compounds can be administered orally. Preferred pharmaceutical compositions may be formulated for oral administration in the form of tablets, capsules, caplets, syrups, solutions, and suspensions. Such oral formulations can be provided in modified release dosage forms such as time-release tablet and capsule formulations. Pharmaceutical compositions can also be administered via injection, namely, intravenously, intramuscularly, subcutaneously, intraperitoneally, intra-arterial, intrathecally, and intracerebroventricularly.

Suitable carriers for injection are well known to those of skill in the art and include 5% dextrose solutions, saline, and phosphate-buffered saline.

According to another aspect, a formulation of the compound of the present invention can be prepared by entrapping the compound in liposomes or albumin. The formulation of the compound of the present invention can be prepared by using nanoparticles, nanocapsules or nanospheres using appropriate excipient(s) such as cyclodextrins, mannitol, sodium dodecyl sulfate, albumin, polysorbate 80, trehalose, sucrose, lactose, tromethamine, sodium chloride, gelatin, amino acids.

In another embodiment of the invention, stabilizing excipients for preparing formulations of a compound of Formula I are selected from hydrophobicity-inducing agents. These agents may be represented by magnesium Stearate, Stearic acid, glyceryl Stearate, glyceryl palmitostearate, Stearoyl macrogolglycerides, lauroyl macrogolglycerides, waxes, and hydrogenated vegetable oils, among others.

The stabilizers may be included into the formulations for the compound of Formula I for the of the current invention in the amount Such that, for an individual stabilizer, the ratio of the parts by weight of stabilizer to parts by weight of the drug substance is from 0.1:1 to 50:1, preferably from 0.25:1 to 40:1; most preferably from 0.4:1 to 25:1. Combinations of stabilizing excipients may be used in all embodiments of the instant invention and may provide synergistic stabilizing action.

Stabilizers may be incorporated into formulations of a compound of Formula I in a variety of ways. They may be intermixed with the drug Substance and/or other excipients or may be provided in the form of a coating on the compound of Formula-containing substrate. Water-based acidifiers may be used in the preparation of the formulations of the current invention as long as care is taken to eliminate or reduce water during the processing. Alternatively, excipients, such as bulking agents, may be pre-treated by the stabilizers prior to their incorporation into the formulation. Stabilization of the compound of Formula I may also be achieved by coating drug layered Substrates with coating polymers dissolved or dispersed in an acidic solution. These and further ways of using stabilizers are disclosed in more detail in the examples below. Additional excipients that can be used alone or in combination to formulate stable compounds of Formula I drug products in accordance with the current invention include bulking agents. Such as lactose anhydrous or lactose monohydrate, (i.e., Supertab 21AN, Ludipress, Ludipress LCE, Fast Flo Lactose, Supertose, Pharmatose, Respitose), glyceryl behenate, hypromellose, ascorbic acid, benzoic acid, carbomer, low moisture microcrystalline cellulose (Avicel® grades PH-103, PH-112, PH-113, PH-200), colloidal silicon dioxide, dextrose (anhydrous), dextrose (anhydrous), maltol, fructose, glyceryl palmitostearate, glyceryl monostearate, guar gum, lactitol (anhydrous), magnesium carbonate, maltitol, maltose, mannitol, polyethylene oxide, Sorbitol. Sucrose, compressible Sugar, confectioner's Sugar, Xylitol; glidants such as talc, starch, and colloidal silicon dioxide and the metallic Stearates; lubricants selected from talc, sodium Stearyl fumarate, hydrogenated vegetable oils, glyceryl palmitostearate, glyceryl behenate, poloxamer, Stearic acid, Stearyl alcohol, cetyl alcohol, waxes, and the metallic Stearates; wetting and solubility enhancing agents, such as sodium lauryl Sulfate, polyethylene glycol, PEG glyceryl esters, lecithin, poloxamer, the polysorbates, the polyoxyethylene alkyl ethers, polyethylene castor oil derivatives, polyethylene Stearate, and the Sorbitan esters. Through the use of stabilizers and low levels of moisture as described above, the inventors were able to realize one goal of the current invention: to provide stable IR formulations of a compound of Formula I that comprise not more than 5% of water. In yet further embodiment, the invention discloses stable IR formulations of a compound of Formula I comprising stabilizing excipients. A further goal of the current invention is to utilize stabilization techniques described herein to provide stable MR formulations of a compound of Formula I comprising an active compound, at least one release controlling polymer that may be a non-pH-dependent polymer or a pH-dependent, enteric polymer, and at least one pharmaceutically acceptable excipient.

Further, the invention provides MR formulations of a compound of Formula I comprising a compound of Formula I, at least one release controlling polymer, and at least one pharmaceutically acceptable excipient, wherein the total amount of residual water in the formulation is not more than 5% by weight of the formulation. The MR formulations of a compound of Formula I exhibiting XR profile, or combination of XR and DR profile, or any combination of those with IR profile are disclosed herein. These specific release profiles are achieved by formulating a compound of Formula I, at least one release controlling polymer, and one or more excipients in a variety of inventive formulations. The release controlling polymers of the current invention may be selected from non-pH-dependent polymers such as hydrophilic rate controlling compound that can be used to formulate MR multi-particulates or matrix tablets drug products, and hydrophobic rate-controlling compounds that exhibit limited or no water solubility; or enteric polymers that exhibit pH-dependent solubility.

Osmotic tablets can be formulated as a single or as a multiple-layer core. In one embodiment, the osmotic tablet comprises a bilayer core, wherein one layer comprises agents to modulate drug release, such as a solubilizer, that are released in a Sustained manner, and the second layer comprises the drug and potentially other agents to modulate drug release. Stabilizers listed above may be contained in at least one layer of the osmotic formulation. An overcoat of the drug can be applied to the osmotic tablet following a functional coating to provide an immediate release component to the dosage form. Alternatively, the osmotic tablet may be coated with an enteric polymer on top of the semipermeable rate-controlling membrane providing a DR/XR profile.

Pharmaceutical compositions may also be administered using other means, for example, rectal administration. Formulations useful for rectal administration, such as suppositories, are well known to those of skill in the art. The compounds can also be administered by inhalation, for example, in the form of an aerosol; topically, such as in lotion form; transdermally, such as using a transdermal patch (for example, by using technology that is commercially available from Novartis and Alza Corporation); by powder injection; or by buccal, sublingual, or intranasal absorption. In addition, pharmaceutical compositions may be formulated in unit dose form or in multiple or subunit doses.

The administration of the pharmaceutical compositions described herein can be intermittent, or at a gradual, continuous, constant, or controlled rate. The pharmaceutical compositions may be administered to a warm-blooded animal, for example, a mammal such as a human being. In addition, the time of day and the number of times per day that the pharmaceutical composition is administered can vary.

The compounds, as provided herein, may also be used for the preparation of a medicament for the treatment or prevention of a disease or condition by inhibiting MetAP2. Methods for treating, preventing, delaying the onset of, or slowing the progression of disorders mediated by MetAP2 involved in the regulation or dysregulation of gene expression in mammals in need of such treatment are also provided. The methods involve administering to a subject a therapeutically effective amount of a compound as provided herein, including a salt thereof or a pharmaceutical composition that includes such compounds.

According to one embodiment of the invention the methods for treating, preventing, delaying the onset of, or slowing the progression of disorders mediated by acetylated proteins involved in the regulation or dysregulation of gene expression in mammals in need of such treatment include the administration of at least one compound as provided herein including, but not limited to, the compounds provided according to Formula I.

The compounds alone or in a pharmaceutical composition as provided herein may be used in the treatment of a variety of disorders and conditions and, as such, may be used in combination with a variety of other suitable therapeutic agents useful in the treatment or prophylaxis of those disorders or conditions. Thus, one embodiment of the present disclosure includes the administration of the compound of the present disclosure in combination with other therapeutic compounds. Such a combination of pharmaceutically active agents may be administered together or separately, and, when administered separately, the administration may occur simultaneously or sequentially, in any order. The amounts of the compounds or agents and the relative timings of administration will be selected in order to achieve the desired therapeutic effect. The administration in a combination of a compound of the present disclosure with other treatment agents may be in combination by administration concomitantly in: (1) a unitary pharmaceutical composition including two or more compounds; or (2) separate pharmaceutical compositions, each including one of the compounds. Alternatively, the combination may be administered separately in a sequential manner wherein one treatment agent is administered first and the other second. Such sequential administration may be close in time or remote in time.

Another aspect of the present disclosure includes combination therapy comprising administering to the subject a therapeutically or prophylactically effective amount of the compound of the present disclosure and one or more other therapy, including chemotherapy, radiation therapy, gene therapy, or immunotherapy.

### DETAILED DESCRIPTION OF THE INVENTION

The present invention relates to compounds that are inhibitors of methionine aminopeptidase 2 (MetAP2), to process of preparing these compounds, their salts, prodrugs, and metabolites, pharmaceutical compositions containing these compounds, and methods of using these compounds for treating a wide variety of medical conditions, diseases or disorders. In one aspect, the invention provides compounds of Formula I, or its pharmaceutically acceptable salt or a prodrug or metabolite(s) thereof

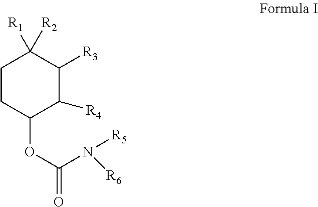

wherein
R₁ is selected from a group of —CH₂Cl, and —CH₂Br;
R₂ is selected from a group of hydrogen and hydroxyl;
R₁ and R₂ together can form

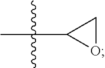 [SMILES: *C1CO1]

R₃ is selected from a group of hydrogen and substituted or unsubstituted alkyl;
R₄ is selected from a group of hydrogen and alkoxy;
R₅ is selected from a group of hydrogen and alkyl; and
R₆ is selected from a group of substituted or unsubstituted alkyl boronic acids, substituted or unsubstituted alkyl boronic acid esters, substituted or unsubstituted aryl boronic acids, substituted or unsubstituted aryl boronic acid esters, substituted or unsubstituted heterocyclic boronic acids, substituted or unsubstituted heterocyclic boronic acid esters;
NR₅R₆ can form a substituted or unsubstituted heterocyclic boronic acids, substituted or unsubstituted heterocyclic boronic acid esters.

According to one embodiment of the invention, the invention also provides a compound of Formula I, wherein
R₁ and R₂ together can form

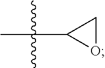 [SMILES: *C1CO1]

According to one embodiment of the invention, the invention also provides a compound of Formula I wherein
R₃ is selected from a group of

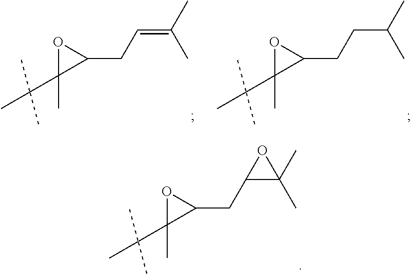

According to one embodiment of the invention, the invention also provides a compound of Formula I wherein R₄ is methoxy

According to one embodiment of the invention, the invention also provides a compound of Formula I wherein R₅ is selected from a group of hydrogen, methyl, and ethyl.

According to one embodiment of the invention, the invention also provides a compound of Formula I wherein R₆ is selected from a group of substituted or unsubstituted alkyl boronic acids, substituted or unsubstituted alkyl boronic acid esters, substituted or unsubstituted aryl boronic acids, substituted or unsubstituted aryl boronic acid esters, substituted or unsubstituted heterocyclic boronic acids, and substituted or unsubstituted heterocyclic boronic acid esters.

According to one embodiment of the invention, the invention also provides a compound of Formula II 1

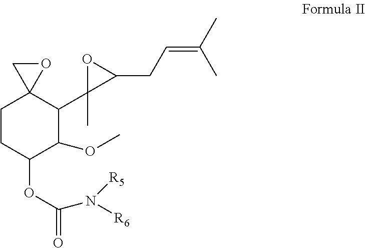

wherein
R₅ is selected from a group of hydrogen and alkyl; and
R₆ is selected from a group consisting of:

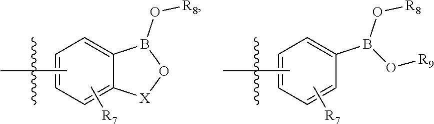

### Wherein

R₇ is one or more substituent selected from a group of hydrogen, amino acid, halogen, alkylthio, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each is optionally substituted;
R₈ and R₉ are independently selected from a group of hydrogen, substituted or unsubstituted alkyl and
R₈ and R₉ can form a moiety derived from a dihydroxy compound having at least two hydroxy groups separated by at least two connecting atoms in a chain or ring, said chain or ring comprising carbon atoms, and optionally, a heteroatom or heteroatoms which can be N. S. or O; and
X is (CR₁₀R₁₁)ₙ wherein R₁₀ and R₁₁ are independently selected from hydrogen and alkyl, and
n=1 and 2.

According to one embodiment of the invention, the invention also provides a compound of Formula III

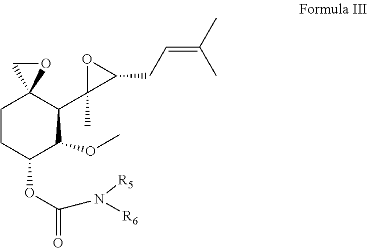

wherein
R₅ is selected from a group of hydrogen and alkyl; and
R₆ is selected from a group of substituted or unsubstituted aryl boronic acid esters, substituted or unsubstituted heterocyclic boronic acids, and substituted or unsubstituted heterocyclic boronic acid esters.

According to one embodiment of the invention, the invention also provides a compound of Formula IV

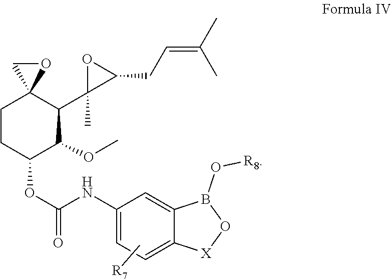

### Wherein

R₇ is one or more substituent selected from a group of hydrogen, amino acid, halogen, alkylthio, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each is optionally substituted;
R₃ is selected from a group of hydrogen, substituted or unsubstituted alkyl and X is (CR₁₀R₁₁)ₙ wherein R₁₀ and R₁₁ are independently selected from hydrogen and alkyl, and
n=1 and 2.

According to one embodiment of the invention, the invention also provides a compound of Formula V

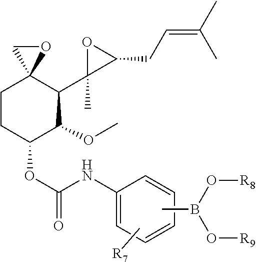

### Wherein

R₇ is one or more substituent selected from a group of hydrogen, amino acid, halogen, alkylthio, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each is optionally substituted;
R₈ and R₉ are independently selected from a group of hydrogen, substituted or unsubstituted alkyl and R₈ and R₉ can form a moiety derived from a dihydroxy compound having at least two hydroxy groups separated by at least two connecting atoms in a chain or ring, said chain or ring comprising carbon atoms, and optionally, a heteroatom or heteroatoms which can be N. S. or O; and

According to one embodiment of the invention, the invention also provides a compound of Formula VI

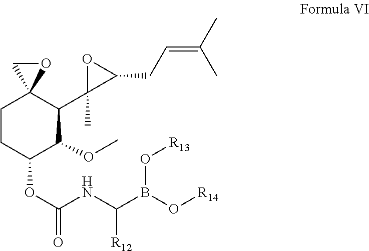

### Wherein

R₁₂ is selected from a group of hydrogen, methyl, ethyl, propyl, isopropyl, butyl, isobutyl, pheny, benzyl, and
R₁₃ and R₁₄ are independently selected from a group of hydrogen, substituted or unsubstituted alkyl and
R₁₃ and R₁₄ can form a moiety derived from a dihydroxy compound having at least two hydroxy groups separated by at least two connecting atoms in a chain or ring, said chain or ring comprising carbon atoms, and optionally, a heteroatom or heteroatoms which can be N. S. or O.

According to one embodiment of the invention, a method of use of a compound of Formula I as defined in the claim 1 for therapeutic use in patient.

According to one embodiment of the invention, a pharmaceutical composition comprising a compound of Formula I or a pharmaceutically acceptable salt, ester, or prodrug or metabolite thereof in association with a pharmaceutically acceptable diluent or carrier.

According to one embodiment of the invention, a pharmaceutical composition comprising a compound of Formula I or a pharmaceutically acceptable salt, ester or prodrug, or metabolite thereof in association with a pharmaceutically acceptable diluent or carrier to form a formulation system for delivering the compound.

According to one embodiment of the invention, a pharmaceutical composition comprising a compound of Formula I or a pharmaceutically acceptable salt, ester, or prodrug or metabolite thereof in a combination of other pharmaceutically active agent(s).

According to one embodiment of the invention, a compound of Formula I or a pharmaceutically acceptable salt, ester or prodrug or metabolite thereof for use in therapy.

According to one embodiment of the invention, the use of a compound of Formula I or a pharmaceutically acceptable salt, ester or prodrug or metabolite thereof in the preparation of a medicament for the treatment and or prevention of parasitic disease, cancer, obesity, angiogenesis, inflammation, and immune disorders.

According to one embodiment of the invention, a process of producing a compound of Formula I or its pharmaceutically acceptable salt or prodrug or metabolite.

According to one embodiment of the invention, a method for the treatment or prevention of a disease or condition by inhibiting MetAP2 is provided. It includes the step of administering a compound as provided herein. Any of the methods or uses provided herein may include administering to a subject a therapeutically effective amount of a compound of Formula I as provided herein, including salt or polymorph thereof, or a pharmaceutical composition that includes such compounds.

According to one embodiment of the invention, a method of use of compound of Formula I for treating the giardiasis, amebiasis, and a combination thereof.

According to one embodiment of the invention, a method of use of compound of Formula I for treating disease in human.

According to one embodiment of the invention, a method of use of compound of Formula I for treating disease in a cat or dog.

According to one embodiment of the invention, a kit comprising a compound of Formula I including pharmaceutically acceptable prodrugs, metabolites, and isomers thereof

According to one embodiment of the invention, a kit comprising a compound of Formula I including pharmaceutically acceptable prodrugs, metabolites, and isomers, wherein the compound and the pharmaceutically acceptable carrier are in separate containers.

According to one embodiment of the invention, a process to produce a compound of Formula 1.

According to one embodiment of the invention, a process to produce a compound of Formula 1 as described in schemes A-K.

According to one embodiment of the invention, a process to produce compound 1 (Table A) as described in scheme K and examples 1-8.

The invention relates to formulation systems loaded with compounds of this invention mentioned above which said systems have improved biopharmaceutical properties for solubility, drug concentration in the target tissue (s), in vivo efficacy and safety, improved quality (fineness and homogeneity of the particles, drug inclusion) and improved physical stability of the particulate formulation (no aggregation or gel formation). The compound of this invention can be appropriately formulated for desired delivery systems such as oral drug delivery (immediate release, delayed-release, prolonged-release, modified release), parenteral drug delivery, ophthalmic drug delivery, nasal drug delivery, rectal drug delivery, intestinal-specific delivery, colon-specific drug delivery, topical drug delivery, and CNS or brain drug delivery by powder injection; or by buccal, sublingual, or intranasal absorption. Pharmaceutical compositions may be formulated in unit dose form or in multiple or subunit doses.

The manner in which the compounds or their pharmaceutical composition set forth herein may be administered can vary. According to one embodiment of the invention the compounds can be administered orally. Preferred pharmaceutical compositions may be formulated for oral administration in the form of tablets, capsules, caplets, syrups, solutions, and suspensions. Such oral formulations can be provided in modified release dosage forms such as time-release tablet and capsule formulations. Pharmaceutical compositions can also be administered via injection, namely, intravenously, intramuscularly, subcutaneously, intraperitoneally, intra-arterial, intrathecally, and intracerebroventricularly.

Suitable carriers for injection are well known to those of skill in the art and include 5% dextrose solutions, saline, and phosphate-buffered saline.

According to another embodiment of the invention, a formulation of the compound of the present invention can be prepared by entrapping the compound in liposomes or albumin. The formulation of the compound of the present invention can be prepared by using nanoparticles, nanocapsules or nanospheres using appropriate excipient(s) such as cyclodextrins, mannitol, sodium dodecyl sulfate, albumin, polysorbate 80, trehalose, sucrose, lactose, tromethamine, sodium chloride, gelatin, amino acids.

In another embodiment of the invention, stabilizing excipients for preparing formulations of a compound of Formula I are selected from hydrophobicity-inducing agents. These agents may be represented by magnesium Stearate, Stearic acid, glyceryl Stearate, glyceryl palmitostearate, Stearoyl macrogolglycerides, lauroyl macrogolglycerides, waxes, and hydrogenated vegetable oils, among others.

The stabilizers may be included into the formulations for a compound of Formula I for the of the current invention in the amount Such that, for an individual stabilizer, the ratio of the parts by weight of stabilizer to parts by weight of the drug substance is from 0.1:1 to 50:1, preferably from 0.25:1 to 40:1; most preferably from 0.4:1 to 25:1. Combinations of stabilizing excipients may be used in all embodiments of the instant invention and may provide synergistic stabilizing action.

Stabilizers may be incorporated into formulations of a compound of Formula I in a variety of ways. They may be intermixed with the drug Substance and/or other excipients or may be provided in the form of a coating on the compound of Formula I-containing substrate. Water-based acidifiers may be used in the preparation of the formulations of the current invention as long as care is taken to eliminate or reduce water during the processing. Alternatively, excipients, such as bulking agents, may be pre-treated by the stabilizers prior to their incorporation into the formulation. Stabilization of a compound of Formula I may be also achieved by coating drug layered Substrates with coating polymers dissolved or dispersed in an acidic solution. These and further ways of using stabilizers are disclosed in more detail in the examples below. Additional excipients that can be used alone or in combination to formulate stable compounds of Formula I drug products in accordance with the current invention include bulking agents. Such as lactose anhydrous or lactose monohydrate, (i.e., Supertab 21AN, Ludipress, Ludipress LCE, Fast Flo Lactose, Supertose, Pharmatose, Respitose), glyceryl behenate, hypromellose, ascorbic acid, benzoic acid, carbomer, low moisture microcrystalline cellulose (Avicel® grades PH-103, PH-112, PH-113, PH-200), colloidal silicon dioxide, dextrose (anhydrous), dextrose (anhydrous), maltol, fructose, glyceryl palmitostearate, glyceryl monostearate, guar gum, lactitol (anhydrous), magnesium carbonate, maltitol, maltose, mannitol, polyethylene oxide, Sorbitol. Sucrose, compressible Sugar, confectioner's Sugar, Xylitol; glidants such as talc, starch, and colloidal silicon dioxide and the metallic Stearates; lubricants selected from talc, sodium Stearyl fumarate, hydrogenated vegetable oils, glyceryl palmitostearate, glyceryl behenate, poloxamer, Stearic acid, Stearyl alcohol, cetyl alcohol, waxes, and the metallic Stearates; wetting and solubility enhancing agents, such as sodium lauryl Sulfate, polyethylene glycol, PEG glyceryl esters, lecithin, poloxamer, the polysorbates, the polyoxyethylene alkyl ethers, polyethylene castor oil derivatives, polyethylene Stearate, and the Sorbitan esters. Through the use of stabilizers and low levels of moisture as described above, the inventors were able to realize one goal of the current invention: to provide stable IR formulations of a compound of Formula I that comprise not more than 5% of water. In yet further embodiment, the invention discloses stable IR formulations of a compound of Formula I comprising stabilizing excipients. A further goal of the current invention is to utilize stabilization techniques described herein to provide stable MR formulations of a compound of Formula I comprising an active compound, at least one release controlling polymer that may be a non-pH-dependent polymer or a pH-dependent, enteric polymer, and at least one pharmaceutically acceptable excipient.

Further, the invention provides MR formulations of a compound of Formula I comprising a compound of Formula I, at least one release controlling polymer, and at least one pharmaceutically acceptable excipient, wherein the total amount of residual water in the formulation is not more than 5% by weight of the formulation. The MR formulations of a compound of Formula I exhibiting XR profile, or combination of XR and DR profile, or any combination of those with IR profile are disclosed herein. These specific release profiles are achieved by formulating a compound of Formula I, at least one release controlling polymer, and one or more excipients in a variety of inventive formulations. The release controlling polymers of the current invention may be selected from non-pH-dependent polymers such as hydrophilic rate controlling compound that can be used to formulate MR multi-particulates or matrix tablets drug products, and hydrophobic rate-controlling compounds that exhibit limited or no water solubility; or enteric polymers that exhibit pH-dependent solubility.

Osmotic tablets can be formulated as a single or as a multiple-layer core. In one embodiment, the osmotic tablet comprises a bilayer core, wherein one layer comprises agents to modulate drug release, such as a solubilizer, that are released in a Sustained manner, and the second layer comprises the drug and potentially other agents to modulate drug release. Stabilizers listed above may be contained in at least one layer of the osmotic formulation. An overcoat of the drug can be applied to the osmotic tablet following a functional coating to provide an immediate release component to the dosage form. Alternatively, the osmotic tablet may be coated with an enteric polymer on top of the semipermeable rate-controlling membrane providing a DR/XR profile.

Pharmaceutical compositions may also be administered using other means, for example, rectal administration. Formulations useful for rectal administration, such as suppositories, are well known to those of skill in the art. The compounds can also be administered by inhalation, for example, in the form of an aerosol; topically, such as in lotion form; transdermally, such as using a transdermal patch (for example, by using technology that is commercially available from Novartis and Alza Corporation); by powder injection; or by buccal, sublingual, or intranasal absorption. Pharmaceutical compositions may be formulated in unit dose form or in multiple or subunit doses.

The administration of the pharmaceutical compositions described herein can be intermittent, or at a gradual, continuous, constant, or controlled rate. The pharmaceutical compositions may be administered to a warm-blooded animal, for example, a mammal such as a human being. In addition, the time of day and the number of times per day that the pharmaceutical composition is administered can vary.

The compounds, as provided herein, may also be used for the preparation of a medicament for the treatment or prevention of a disease or condition by inhibiting MetAP2. Methods for treating, preventing, delaying the onset of, or slowing the progression of disorders mediated by MetAP2 involved in the regulation or dysregulation of gene expression in mammals in need of such treatment are also provided. The methods involve administering to a subject a therapeutically effective amount of a compound as provided herein, including a salt thereof or a pharmaceutical composition that includes such compounds.

According to one embodiment of the invention the methods for treating, preventing, delaying the onset of, or slowing the progression of disorders mediated by acetylated proteins involved in the regulation or dysregulation of gene expression in mammals in need of such treatment include the administration of at least one compound as provided herein including, but not limited to, the compounds provided according to Formula I.

The compounds alone or in a pharmaceutical composition as provided herein may be used in the treatment of a variety of disorders and conditions and, as such, may be used in combination with a variety of other suitable therapeutic agents useful in the treatment or prophylaxis of those disorders or conditions. Thus, one embodiment of the present disclosure includes the administration of the compound of the present disclosure in combination with other therapeutic compounds. Such a combination of pharmaceutically active agents may be administered together or separately, and, when administered separately, the administration may occur simultaneously or sequentially, in any order. The amounts of the compounds or agents and the relative timings of administration will be selected in order to achieve the desired therapeutic effect. The administration in a combination of a compound of the present disclosure with other treatment agents may be in combination by administration concomitantly in: (1) a unitary pharmaceutical composition including two or more compounds; or (2) separate pharmaceutical compositions, each including one of the compounds. Alternatively, the combination may be administered separately in a sequential manner wherein one treatment agent is administered first and the other second. Such sequential administration may be close in time or remote in time.

Another aspect of the present disclosure includes combination therapy comprising administering to the subject a therapeutically or prophylactically effective amount of the compound of the present disclosure and one or more other therapy, including chemotherapy, radiation therapy, gene therapy, or immunotherapy.

### Definitions

The following definitions are meant to clarify, but not limit, the terms defined. If a particular term used herein is not specifically defined, such term should not be considered indefinite. Rather, terms are used within their accepted meanings.

As used throughout this specification, the preferred number of atoms, such as carbon atoms, will be represented by, for example, the phrase “CₓC_{y} alkyl,” which refers to an alkyl group, as herein defined, containing the specified number of carbon atoms. Similar terminology will apply to other preferred terms and ranges as well. Thus, for example, C₁₋₆ alkyl represents a straight or branched chain hydrocarbon containing one to six carbon atoms.

As used herein, the term “alkyl” refers to a straight or branched chain hydrocarbon, which may be optionally substituted, with multiple degrees of substitution being allowed. The alkyl chain may also have one or more unsaturated bond such as includes one or more carbon-carbon double bonds. The term “lower alkyl” refers to an alkyl that includes one to six carbon atoms. Example of

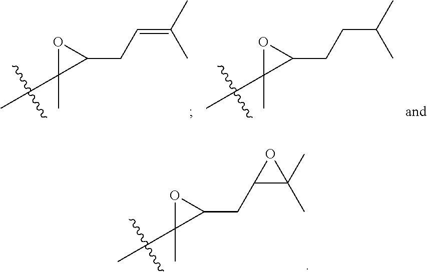

alkyl group also includes

Examples of “lower alkyl” as used herein include, but are not limited to, methyl, ethyl, propyl, isopropyl, isobutyl, n-butyl, tert-butyl, isopentyl, and n-pentyl.

As used herein, the term “alkene” or “alkenyl” group refers to an unsaturated hydrocarbon that includes one or more carbon-carbon double bonds. The term “lower alkene” refers to an alkene that includes from two to twenty carbon atoms, such as from two to ten carbon atoms. The term “substituted alkene” refers to an alkene that has one or more of its hydrogen atoms replaced by one or more substituent groups, such as halogen.

As used herein, the term “alkyne” or “alkynyl” group refers to an unsaturated hydrocarbon that includes one or more carbon-carbon triple bonds. The term “lower alkyne” refers to an alkyne that includes from two to twenty carbon atoms, such as from two to ten carbon atoms. The term “substituted alkyne” refers to an alkyne that has one or more of its hydrogen atoms replaced by one or more substituent groups, such as halogen.

As used herein, the term “cycloalkyl” refers to a fully saturated optionally substituted monocyclic, bicyclic, or bridged hydrocarbon ring, with multiple degrees of substitution being allowed. Preferably, the ring is three to twelve-membered, more preferably, from five- to six-membered. Exemplary “cycloalkyl” groups as used herein include but are not limited to cyclopropyl, cyclobutyl, cyclopentyl, cyclohexyl, and cycloheptyl.

As used herein, the term “alkoxy” refers to a group —OR^{a}, where R^{a} is “alkyl” as defined herein.

As used herein, the term “heterocycloalkyl” or “heterocycle” or “heterocyclyl” refers to an optionally substituted mono- or polycyclic ring system, optionally containing one or more degrees of unsaturation, and also containing one or more heteroatoms, which may be optionally substituted, with multiple degrees of substitution being allowed. Exemplary heteroatoms include nitrogen, oxygen, or sulfur atoms, including N-oxides, sulfur oxides, and carbon oxides. Preferably, the ring is three to twelve-membered, preferably four, five, or six-membered, and is either fully saturated or has one or more degrees of unsaturation. In addition, such rings may be optionally fused to one or more of another heterocyclic ring(s) or cycloalkyl ring(s). Examples of “heterocyclic” groups as used herein include, but are not limited to, tetrahydrofuran, pyran, tetrahydropyran, 1,4-dioxane, 1,3-dioxane, piperidine, pyrrolidine, morpholine, tetrahydrothiopyran, tetrahydrothiophene, pyrrolidinone, dihydrofuranone, thiazolidinone, azetidinone, cyclopentanone, piperidinone, thiomorpholinone, 2H-1,4-thiazin-3(4H)-one, dihydropyrimidine-2,4(1 H,3H)-dione 1,4-dihydropyridine.

As used herein, the term “aryl” refers to a single benzene ring or fused benzene ring system which may be optionally substituted, with multiple degrees of substitution being allowed. Examples of “aryl” groups as used include, but are not limited to, phenyl, benzyl, 2-naphthyl, 1-naphthyl, anthracene, and phenanthrene. Preferable aryl rings have five- to ten members. The term “aryl” also includes a fused benzene ring system, namely where a cyclic hydrocarbon or heterocycle (e.g., a cyclohexane or dioxane ring) or heteroaryl (e.g., pyridine) is fused with an aromatic ring (aryl, such as a benzene ring).

As used herein, the term “heteroaryl” refers to a monocyclic five to seven-membered aromatic ring, a fused bicyclic aromatic ring system comprising two of such aromatic rings, which may be optionally substituted, with multiple degrees of substitution being allowed, or to a fused bicyclic ring system namely where a cycloalkyl or heterocycle (e.g., a cyclohexane or dioxane ring) is fused with a heteroaryl ring. Preferably, heteroaryl rings contain five- to ten-members. These heteroaryl rings contain one or more nitrogen, sulfur, and/or oxygen atoms. In certain embodiments, the heteroaryl rings contain one to three nitrogen, one to three oxygen, or one or two sulfur atoms. N-oxides, sulfur oxides and dioxides are permissible heteroatom substitutions. Examples of “heteroaryl” groups as used herein include, but are not limited to, furan, thiophene, pyrrole, imidazole, pyrazole, triazole, tetrazole, thiazole, oxazole, isoxazole, oxadiazole, thiadiazole, isothiazole, triazole, pyridine, pyridazine, pyrazine, pyrimidine, quinoline, isoquinoline, quinoxaline, benzofuran, benzoxazole, benzothiophene, indole, indazole, benzimidazole, imidazopyridine, pyrazolopyridine, and pyrazolopyrimidine.

As used herein, the term “halogen” refers to fluorine, chlorine, bromine, or iodine.

As used herein, the term “haloalkyl” refers to a substituted or unsubstituted alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein, the term “sulfhydryl” refers to refers to a —SH group.

As used herein, the term “alkylthio” refers to a group —SR^{a}, where R^{a} is “alkyl” as defined herein.

As used herein, the term “arylthio” refers to a group —SR^{a}, where R^{a} is “aryl” as defined herein.

As used herein, the term “carboxyamido” refers to —NH—C(O)—W, wherein W is hydrogen or an unsubstituted or substituted alkyl, alkene, alkyne, cycloalkyl, aryl, or heterocycle group.

As used herein, the term “amine” is given its ordinary meaning and includes primary, secondary, and tertiary amines.

As used herein, the term “amido” refers to a group of the formula —C(O)NR′R′, wherein R′ and R″ are substituted or unsubstituted alkyl, cycloalkyl or heterocycle, or R′ and R″ can form cycloalkyl or heterocycle. As used herein, the term “sulfamido” refers to the group —SO₂NR′R″.

As used herein, the term boronic acid refers to an alkyl or aryl or heteroaryl substituted boric acid containing a carbon to boron chemical bond (RB(OH)2) or hemiacid (RBOH(OR′)). Examples of boronic acid groups as used herein include, but are not limited to following structures,

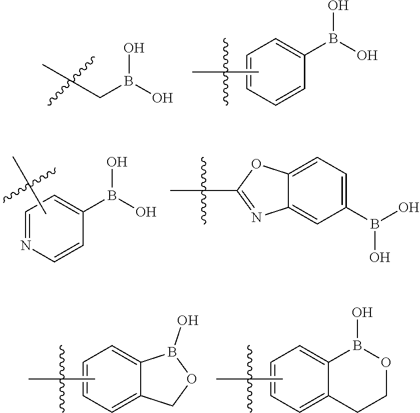

As used herein, the term boronic acid ester refers to an alkyl or aryl or heteroaryl substituted boric acid containing a carbon to boron chemical bond (RB(OR′)2) or hemiester (RBOH(OR′)). Examples of boronic acid groups as used herein include, but are not limited to following structures,

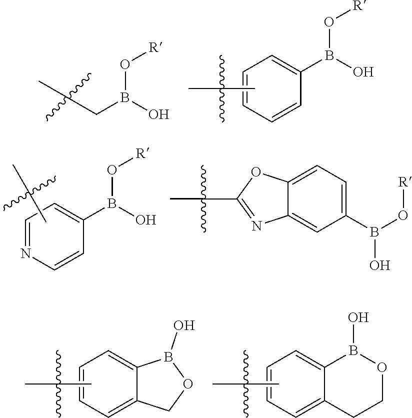

As used herein, “optionally substituted” groups may be substituted or unsubstituted. The substituent (or substitution) group may include, without limitation, one or more substituents independently selected from the following groups or designated subsets thereof: lower (C₁-C₆) alkyl, lower alkenyl, lower alkynyl, lower aryl, heteroaryl, alicyclic, heterocyclic, arylalkyl, heteroarylalkyl, lower alkoxy, lower aryloxy, amino, alkylamino, dialkylamino, diarylalkylamino, alkylthio, arylthio, heteroarylthio, oxo, oxa, carbonyl (—C(O)), carboxy esters (—C(O)OR), carboxamido (—C(O)NH₂), carboxy, acyloxy, —H, halo, —CN, —NO₂, —N₃, —SH, —OH, —C(O)CH₃, perhaloalkyl, perhaloalkoxy, perhaloacyl, guanidine, pyridinyl, thiophene, furanyl, indole, indazole, esters, amides, phosphonates, phosphonic acid, phosphates, phosphoramides, sulfonates, sulfones, sulfates, sulphonamides, carbamates, ureas, thioureas and thioamides, thioalkyls. An optionally substituted group may be unsubstituted (eg., —CH₂CH₃), fully substituted (e.g., —CF₂CF₃), or monosubstituted (e.g., —CH₂CH₂F) or substituted at a level anywhere in-between fully substituted and monosubstituted (e.g., —CH₂CF₃).

As used herein, the term “pharmaceutically acceptable” refers to the carrier(s), diluent(s), excipient(s), or salt forms of the compounds of the present disclosure that are compatible with the other ingredients of the formulation of the pharmaceutical composition.

As used herein, the term “pharmaceutical composition” refers to a compound of the present disclosure optionally admixed with one or more pharmaceutically acceptable carriers, diluents, or excipients. Pharmaceutical compositions preferably exhibit a degree of stability to environmental conditions to make them suitable for manufacturing and commercialization purposes.

As used herein, the terms “effective amount”, “therapeutic amount”, and “effective dose” refer to an amount of the compound of the present disclosure sufficient to elicit the desired pharmacological or therapeutic effects, thus resulting in effective prevention or treatment of a disorder. Treatment of a disorder may be manifested by delaying or preventing the onset or progression of the disorder, as well as delaying or preventing the onset or progression of symptoms associated with the disorder. Treatment of a disorder may also be manifested by a decrease or elimination of symptoms, reversal of the progression of the disorder, as well as any other contribution to the well-being of the patient. The effective dose can vary, depending upon factors such as the condition of the patient, the severity of the symptoms of the disorder, and the manner in which the pharmaceutical composition is administered.

The term “prodrug” as used herein is intended to encompass a class of analogs of compounds of the present invention wherein a metabolically labile moiety is attached to said compound of the invention through an available NH, C(O)H, COOH, C(O)NH2, OH or SH functionality. The prodrug-forming moieties are removed by metabolic processes and release the active compounds having the free NH, C(O)H, COOH, C(O)NH2, OH, or SH group in vivo.

Prodrugs are useful for adjusting such pharmacokinetic properties of the compounds as solubility and/or hydrophobicity, absorption in the gastrointestinal tract, bioavailability, tissue penetration, and rate of clearance. Design and preparation of such prodrugs are known to those skilled in the art, and are described in: Various forms of prodrugs are well known in the art and are described in:
a) The Practice of Medicinal Chemistry, Camille G. Wermuth et al., Ch. 31 (Academic Press, 1996).
b) Design of Prodrugs, edited by H. Bundgaard, (Elsevier, 1985); 33.
c) A Textbook of Drug Design and Development, P. Krogsgaard-Larson and H. Bundgaard, eds. Ch. 5, pp. 113-191 (Harwood Academic Publishers, 1991); and
d) Hydrolysis in Drug and Prodrug Metabolism, Bernard Testa and Joachim M. Mayer, (Wiley-VCH, 2003).
e) Prodrugs: challenges and rewards, Valentino J. Stella et al., Springer, 2007 Said references are incorporated herein by reference, particularly as to the description of prodrugs.

Chemical names, stereochemistry designations, and chemical properties of compounds of this invention are obtained using Chemoffice tools by Perkin Elmer, and/or Scifinder by American Chemical Society and/or PubChem and/or Wikipedia.

### Synthetic Methods

The compounds of this invention described herein can be prepared by any number of methods known/obvious to those skilled in the art. As a matter of illustration, any of the approaches shown in the following schemes can be used to make such compounds of the formula (1) described herein. Compounds of this invention can be prepared in accordance with one or more of the Schemes discussed below. These procedures of each reaction will be understandable to and can be carried out by someone of ordinary skill in organic chemistry. Starting materials may be prepared according to procedures provided in the references below, or can be prepared by procedures reported in the literature or are commercially available. Unless otherwise defined below, variables used in the Schemes are as defined in the specification or in the claims. PG is a suitable protecting groups (PGs) include, but not limited to, acetyl, Boc, Fmoc, benzoyl, pivaloyl, trityl, tetrahydropyranyl (THP), alkyl ester, and silyl (TBDMS, TMS, etc.). More information about the selection of protecting groups is described in the book “Greene's Protective Groups in Organic Synthesis” 5th Edition, by Peter G. M. Wuts, Publisher: John Wiley & Sons. LG is a suitable leaving group such as a halide, imidazole, O-succinimide, 4-nitrophenoxide, ester, and the like, which can be reacted with the nucleophilic group of the linker in the absence or presence of a suitable base and solvent. More examples and preparation are described in the book Organic Chemistry (8th Edition) by L. G. Wade Jr, Publisher: Pearson.

The processes described herein can be monitored according to any suitable method known in the art. For example, product formation can be monitored by spectroscopic means, such as nuclear magnetic resonance spectroscopy (e.g., 1H or 13C), infrared spectroscopy, spectrophotometry (e.g., UV-visible), mass spectrometry, or by chromatography such as high-performance liquid chromatography (HPLC), gas chromatography (GC), gel-permeation chromatography (GPC), or thin-layer chromatography (TLC).

The reactions or the processes described herein can be carried out in suitable solvents, which can be readily selected by one skilled in the art. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

Compounds of claim 1 of the current invention can be prepared according to Schemes A-J as set forth below.

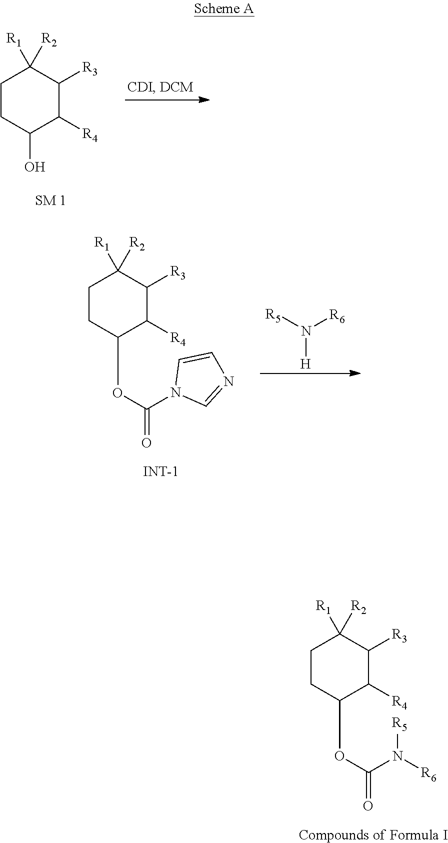

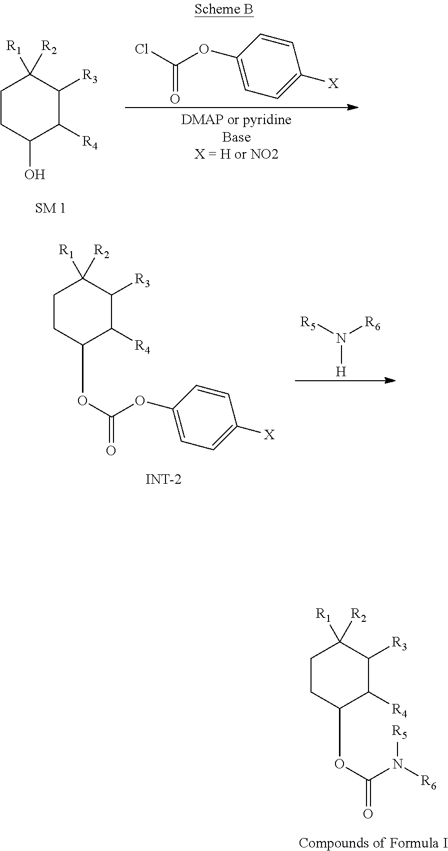

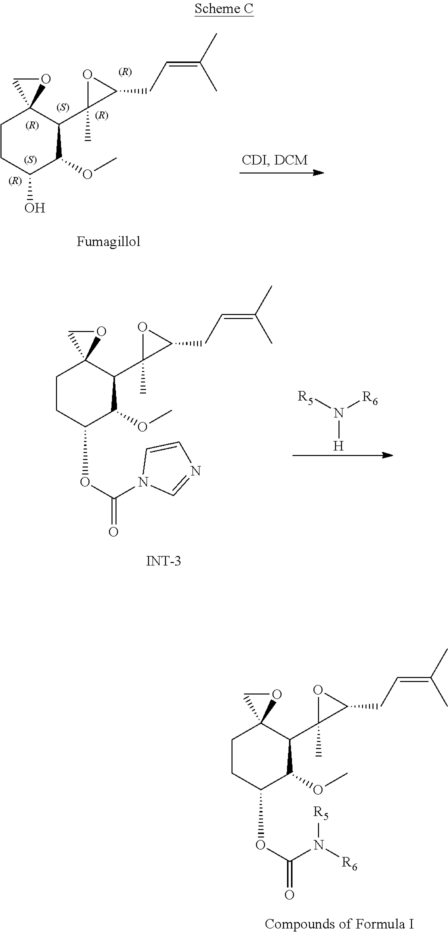

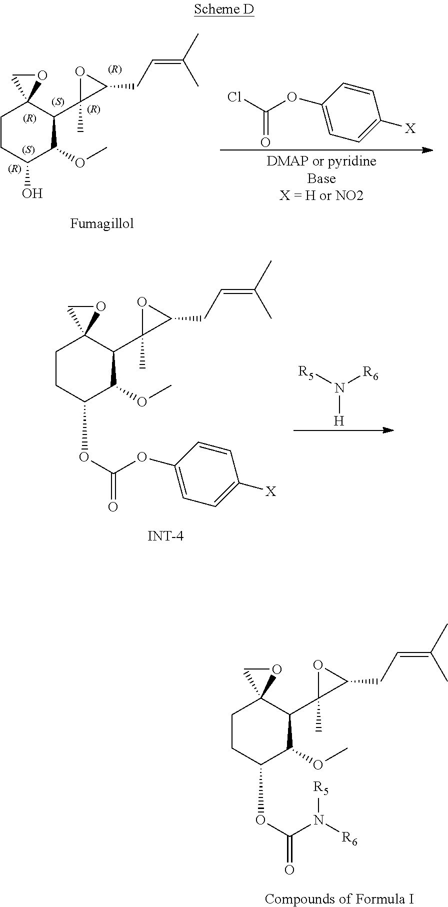

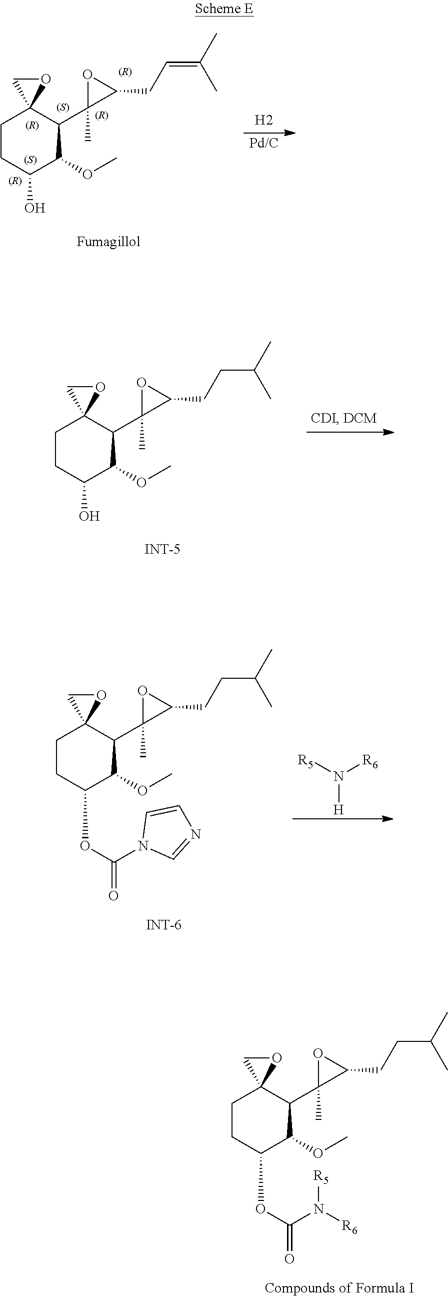

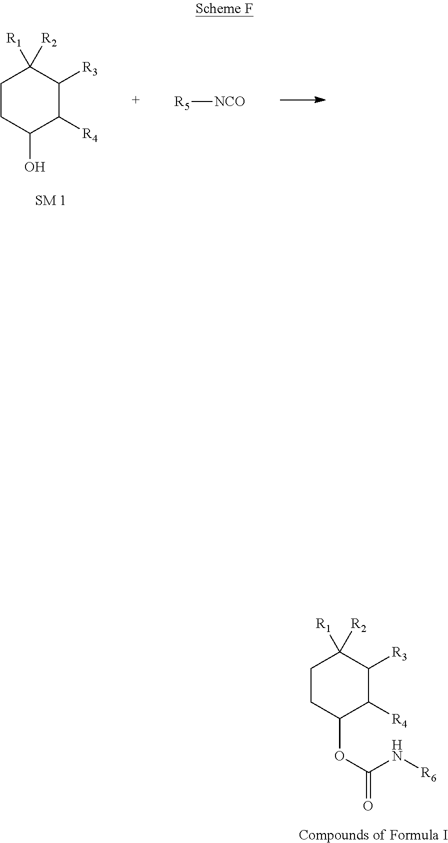

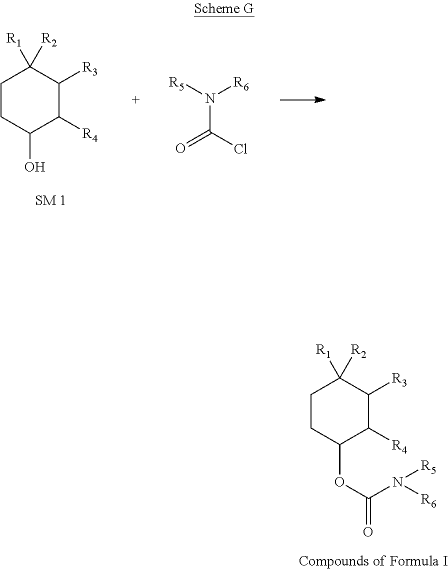

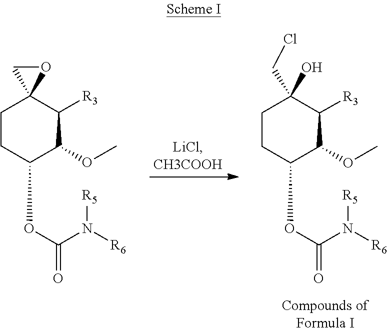

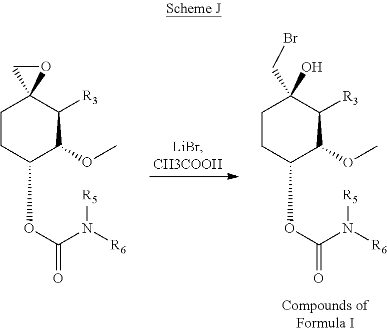

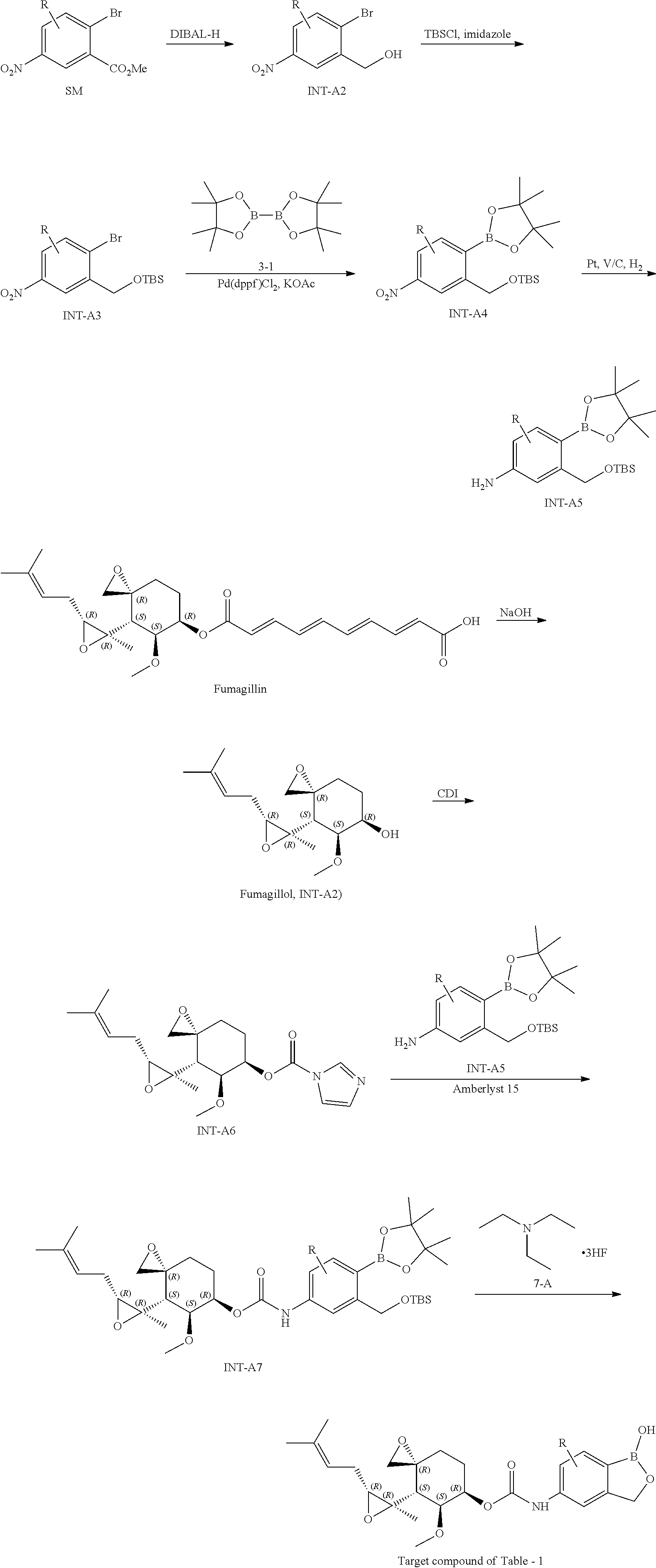

Note that functionality(ies) of substituent(s) on R, R₁, R₂, R₃, R₄, R₅, and R₆ groups in the above schemes may be appropriately protected for synthetic feasibility that can be deprotected to obtain the desired targeted compounds of claim 1. The targeted therapeutic agent may have more than one functional group; in those cases, the other functional group(s) may be protected by appropriate protecting group(s), and they can be deprotected to obtain the desired targeted compound.

The compounds of Table A can be prepared by following the any of the reaction schemes mentioned above

### Experimental Procedures

### Example 1

### Procedure for preparation of fumagillol (Intermediate 1)

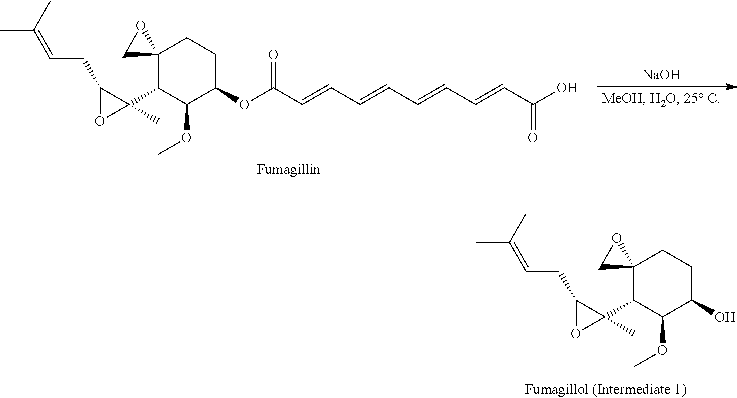 [SMILES: CO[C@@H]1[C@H](OC(=O)/C=C/C=C/C=C/C=C/C(=O)O)CC[C@]2(CO2)[C@H]1[C@@]1(C)O[C@@H]1CC=C(C)C ; "NaOH" ; CO[C@@H]1[C@H](O)CC[C@]2(CO2)[C@H]1[C@@]1(C)O[C@@H]1CC=C(C)C]

A solution of NaOH (116.5 g, 1.02 mol, 3.01 eq) in H₂O (1870 mL) was added dropwise to a mixture of compound Fumagillin (187 g, 339 mmol, 1.00 eq.) in MeOH (1870 mL) at 0° C. The mixture was stirred at 0° C. for 1 hrs under N₂. Then the mixture was stirred at 25° C. for 5 hrs under N₂. TLC (Petroleum ether: Ethyl acetate=1:1) showed compound Fumagillin (Rf=0.47) was consumed, and one new spot (Rf=0.34) was detected. HPLC showed ˜0.813% of the compound Fumagillin (Rt=1.869 min) remained, and several new peaks were detected. The mixture was poured into H₂O (1500 mL), extracted with EA (2000 mL*2). The combined organic layers were dried over Na₂SO₄, filtered, and concentrated under reduced pressure to give a residue. The residue was purified by column chromatography (SiO₂, Petroleum ether/Ethyl acetate=100/1 to 1/1). Fumagillol (Intermediate 1) was obtained as a yellow oil. HNMR confirmed the product.

¹H NMR: 400 MHz CDC₃

δ 0.86-0.88 (dt, 1H), 1.08 (s, 3H), 1.69 (s, 3H), 1.71 (s, 4H), 1.85-1.88 (d, 1H), 2.00-2.03 (td, 1H), 2.16-2.19 (m, 2H), 2.48-2.51 (m, 1H), 2.82 (d, 1H), 3.30 (s, 3H), 3.32-3.38 (d, 2H), 4.55-4.56 (d, 1H), 5.17-5.19 (t, 1H).

### Example 2: Procedure for preparation of (2-bromo-5-nitrophenyl)methanol, (Intermediate 2)

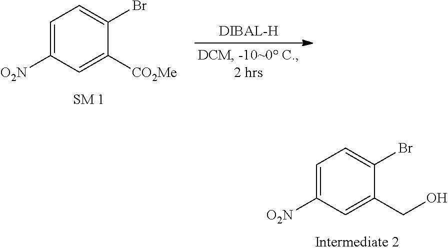 [SMILES: COC(=O)c1cc([N+](=O)[O-])ccc1Br ; O=[N+]([O-])c1ccc(Br)c(CO)c1]

A mixture of compound SM1 (130 g, 500 mmol, 1.00 eq.) in DCM (2000 mL) was cooled down to ˜10-0° C. under N₂ atmosphere. DIBAL-H (1 M, 1.00 L, 2.00 eq.) was added dropwise at−10-0° C. for 1 hrs under N₂ atmosphere. The mixture was stirred at 0° C. for 2 hrs under N₂ atmosphere. H₂O (500 mL) was added, causing a yellow precipitate to form.1 M aqueous HCl (500 mL) was added dropwise, and the reaction was stirred for 30 min at 0° C. The mixture was separated, and the organic phase 1 and aqueous phase 1 was obtained. The aqueous phase 1 was extracted with DCM (1000 ml), and the organic phase was obtained. The combined organic phase was washed with water (1000 ml), and dried over 50 g of anhydrous Na₂SO₄. The organic phase was concentrated under reduced pressure to remove the solvent until no obvious distillate was found. The crude product was triturated with PE/EA=10/1(500 ml) at 25° C. for 10 min to give Intermediate 2 (227 g, 965 mmol, 96.5% yield, 98.6% purity) as a light yellow solid. HNMR and LCMS confirmed the product.

¹H NMR: 400 MHz CDC₃

δ 8.43 (d, 1H), 8.02 (dd, 1H), 7.72 (d, 1H), 4.83 (s, 2H), 2.03-2.36 (m, 1H).

### Example 3: Procedure for preparation of ((2-bromo-5-nitrobenzyl)oxy)(tert-butyl)dimethylsilane (Intermediate 3)

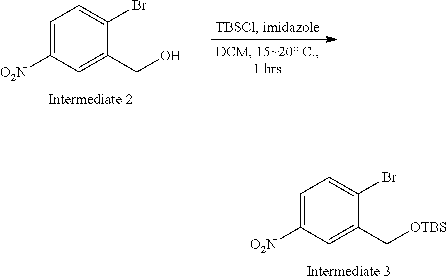 [SMILES: O=[N+]([O-])c1ccc(Br)c(CO)c1 ; CC(C)(C)[Si](C)(C)OCc1cc([N+](=O)[O-])ccc1Br]

A mixture of Intermediate 2 (227 g, 978 mmol, 1.00 eq.) in DCM (1500 mL) was added imidazole (79.9 g, 1.17 mol, 1.20 eq.) and TBSCI (147.5 g, 978 mmol, 120.4 mL, 1.00 eq.) at 25° C. under N₂ atmosphere. The mixture was stirred at 25° C. for 1 hrs. The mixture was washed with citric acid/H₂O (1000 ml) to adjust the pH=3. The mixture was separated, and the organic phase was washed with NaHCO₃/H₂O (500 ml) to adjust the pH=7. The mixture was separated, and the organic phase was washed with sat. Brine (500 mL) was dried over 10 g of Na2SO4, filtered and concentrated in a vacuum. The residue was purified by column chromatography (SiO2, Petroleum ether/Ethyl acetate=100/1 to 10/1). TLC (Petroleum ether/Ethyl acetate=10/1), Rf (P1)=0.80) to give Intermediate 3 (345 g, 954 mmol, 97.6% yield, 95.8% purity), as a yellow oil, confirmed by HNMR and LC-MS.

¹H NMR: 400 MHz CDC₃

δ 8.44 (d, 1H), 7.99 (dd, 1H), 7.67 (d, 1H), 4.76 (s, 2H), 1.00 (s, 8H), 0.18 (s, 6H).

### Example 4: Procedure for preparation of tert-butyldimethyl((5-nitro-2-(4,4,5,5-tetramethyl-1,3,2-dioxaborolan-2-yl)benzyl)oxy)silane (Intermediate 4)

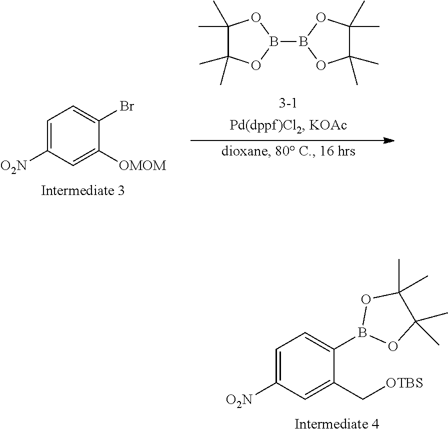

Reagent 3-1 (266 g, 1.05 mol, 1.10 eq.) and KOAc (234 g, 2.38 mol, 2.50 eq.) were added to a mixture of Intermediate 3 (330 g, 953 mmol, 1.00 eq.) in dioxane (2500 mL) at 25° C. under N₂ atmosphere. The mixture was added Pd(dppf)Cl₂ (34.9 g, 47.7 mmol, 0.05 eq.) at 80° C. Then the mixture was stirred at 80° C. for 16 hrs under N2 atmosphere. LC-MS showed ˜46.2% with desired mass (RT=0.809 min, M+H=394). The mixture was cooled to 25° C., H₂O (1000 mL) was added and filtered, the cake was washed with MTBE (1000 mL), and the filtrate was left to stand for 15 min. The mixture was separated, and the organic phase 1 and aqueous phase 1 was obtained. The aqueous phase 1 was extracted with MTBE (500 ml), and the organic phase 2 was obtained. The combined organic phases were washed with brine (500 ml), and dried over 50 g of anhydrous Na₂SO₄. The organic phase was concentrated under reduced pressure to remove the solvent until no obvious distillate was found. The residue was purified by column chromatography (SiO2, Petroleum ether/Ethyl acetate=100/1 to 10/1). TLC (Petroleum ether/Ethyl acetate=10/1), Rf (P1)=0.80) to give the product. The crude product was re-purified by crystallization from PE (600 mL) at −30° C. to give Intermediate 4 (212 g, 532 mmol, 55.8% yield, 98.7% purity) as a yellow solid, confirmed by HNMR and LC-MS.

¹H NMR: 400 MHz CDC₃

δ 8.44-8.48 (m, 1H), 8.04 (dd, 1H), 7.92 (d, 1H), 5.05 (s, 2H), 1.37 (s, 12H), 0.99 (s, 9H), 0.14 (s, 6H).

### Example 5: Procedure for preparation of 3-(((tert-butyldimethylsilyl)oxy)methyl)-4-(4,4,5,5-tetramethyl-1,3,2-dioxaborolan-2-yl)-aniline (Intermediate 5)

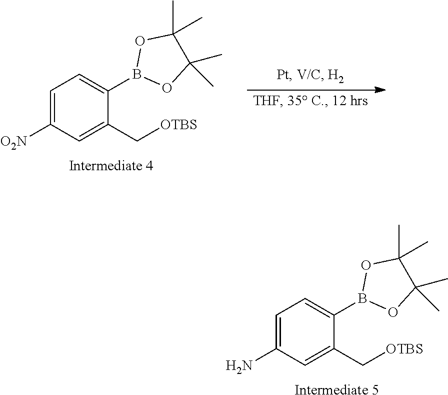

A mixture of compound 4 (120 g, 305 mmol, 1.00 eq.) in THF (1200 mL) was degassed and purged with N₂ at 25° C. for 3 times. Pt, V/C (25 g, 381 mmol) was added at 25° C. under N₂ atmosphere. The reaction was degassed under vacuum and purged with H₂ 3 times. And then, the mixture was stirred at 35° C. for 12 hrs under H₂ atmosphere (50 psi). LC-MS showed ˜98.0% with desired mass (RT=0.826 mins, M+H=364) was detected. The mixture was filtered with 50 g of diatomite; the cake was washed with THF(200 ml×2); the filtrate was collected, dry with 50 g Na₂SO₄, filtered, and concentrated under reduced pressure to give compound 5 (107 g, 292 mmol, 95.8% yield, 99.2% purity), as a brown oil, confirmed by HNMR and LC-MS.

¹H NMR: 400 MHz CDCl₃

δ 7.61 (d, 1H), 6.93-6.98 (m, 1H), 6.52 (dd, 1H), 4.98 (s, 2H), 1.31 (s, 12H), 0.98 (s, 9H), 0.12 (s, 6H).

### Example 6: Procedure for preparation of (3R,4S,5S,6R)-5-methoxy-4-((2R,3R)-2-methyl-3-(3-methylbut-2-en-1-yl)oxiran-2-yl)-1-oxaspiro[2.5]octan-6-yl 1H-imidazole-1-carboxylate (Intermediate 6)

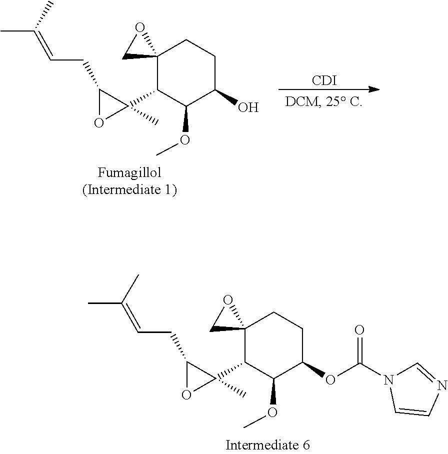

A reagent CDI (64.8 g, 399 mmol, 1.10 eq.) was added to a mixture of Intermediate 1b (102.6 g, 363 mmol, 1.00 eq.) in DCM (1000 mL) under N2 atmosphere. The mixture was stirred at 0° C. for 1 hrs. TLC (Petroleum ether: Ethyl acetate=1:1) showed Intermediate 1b (Rf=0.47) was consumed, and one new spot (Rf=0.40) was detected. The mixture was added H₂O (1200 mL) and extracted with DCM (800 mL*2). The combined organic layers were dried over Na₂SO₄, filtered, and concentrated under reduced pressure to give a residue. Intermediate 1 (140 g, crude) was obtained as a yellow oil, and LCMS confirmed the product.

### Example 7: Procedure for preparation of (3R,4S,5S,6R)-5-methoxy-4-((2R,3R)-2-methyl-3-(3-methylbut-2-en-1-yl)oxiran-2-yl)-1-oxaspiro[2.5]octan-6-yl (3-(((tert-butyldimethylsilyl)oxy)methyl)-4-(4,4,5,5-tetramethyl-1,3,2-dioxaborolan-2-yl)phenyl)carbamate (Intermediate 7)

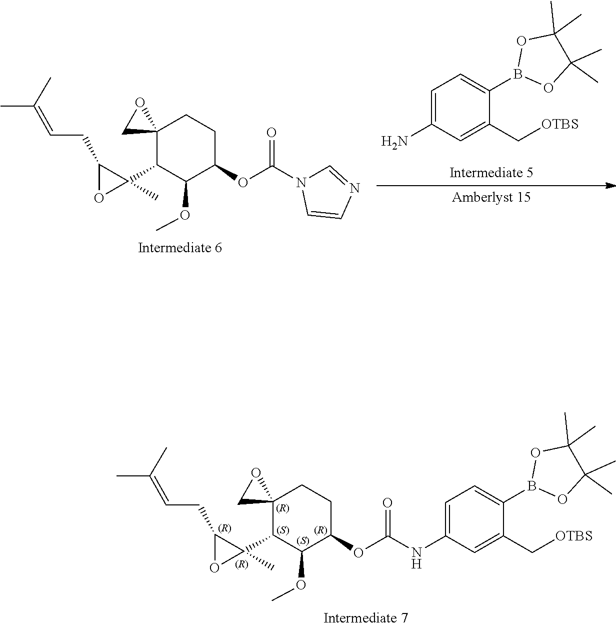

The intermediate 5 (132 g, 363 mmol, 1.00 eq) and Amberlyst 15 (114 g, 363 mmol, 1.00 eq.) were added to a mixture of Intermediate 6 (137 g, 363 mmol, 1.00 eq.) in ACN (1330 mL) under N₂ atmosphere. The mixture was stirred at 50° C. for 12 hrs under N₂ atmosphere. LCMS showed ˜12.4% of Intermediate 6 was remained, ˜58.6% of Intermediate 7 desired mass (RT=0.813 mins), and ˜5.63% of Intermediate 7 desired mass (RT=0.742 min, M+1=546.4) were detected. The reaction mixture was filtered and concentrated to give a residue. The residue was purified by reversed-phase HPLC (Neutral). Intermediate 7 (90.7 g, 129 mmol, 35.6% yield, 95.9% purity) was obtained as a yellow oil and was confirmed by LCMS. Intermediate 7 was precipitated as a white solid and was confirmed by HPLC, LCMS, and HNMR.

LCMS: RT=0.838 mins, M+H⁺: 540.1

HPLC: RT=1.91 mins, Purity=98.1%

¹H NMR: 400 MHz DMSO-d₆

δ 9.73 (s, 1H), 7.65 (s, 1H), 7.55 (d, 1H), 7.41 (dd, 1H), 5.45 (d, 1H), 5.16-5.25 (m, 1H), 4.90 (s, 2H), 3.61 (dd, 1H), 3.33 (s, 3H), 2.87 (d, 1H), 2.60 (d, 1H), 2.52-2.57 (t, 2H), 2.15-2.25 (q, 2H), 1.85-2.05 (m, 3H), 1.68-1.83 (m, 5H), 1.62 (s, 3H), 1.27 (s, 12H), 1.11 (s, 3H), 0.91 (s, 9H), 0.06 (d, 6H).

### Example 8: Procedure for preparation of (3R,4S,5S,6R)-5-methoxy-4-((2R,3R)-2-methyl-3-(3-methylbut-2-en-1-yl)oxiran-2-yl)-1-oxaspiro[2.5]octan-6-yl (1-hydroxy-1,3-dihydrobenzo[c][1,2]oxaborol-5-yl)carbamate (Compound 1) (Table A)

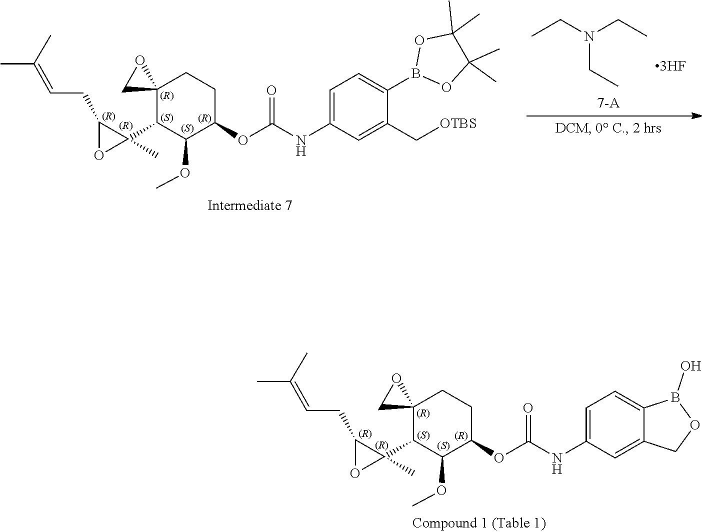

The reagent 7-A (53.3 g, 330 mmol, 53.9 mL, 2.00 eq.) was added to a mixture of Intermediate 7 (111 g, 165 mmol, 1.00 eq.) in DCM (1110 mL) under N₂ atmosphere. The mixture was stirred at 0° C. for 4 hrs under N₂ atmosphere. HPLC showed Intermediate 7 was consumed, and 93.3% of one new peak (RT=1.649 mins) was detected. LCMS showed ˜83.4% of desired mass (RT=0.542 min, M+1=458.3) was detected. The mixture was added NaHCO₃/H₂O to adjust pH to 7. The mixture was added water (1000 mL), extracted twice with DCM (1000 mL). The combined organic layers were dried over Na₂SO₄, filtered, and concentrated under reduced pressure to give a residue. The crude product was purified by reversed-phase HPLC (column: Phenomenex luna C18 250 mm*100 mm*10 um; mobile phase: [A: water (NH₄HCO₃) B: ACN]; gradient: 36%-65% B over 20 min). The final Compound 1 (Table A) (60.07 g, 121.93 mmol, 73.8% yield, 99.8% purity) was obtained as a white solid. HNMR showed the desired compound detected.

LCMS: RT=0.542 mins, M+H⁺: 458.3

HPLC: RT=1.640 mins, Purity=99.8%

¹H NMR: 400 MHz DMSO-d₆

δ 9.76 (s, 1H), 9.00 (s, 1H), 7.61 (d, 2H), 7.37 (d, 1H), 5.46 (d, 1H), 5.20 (t, 1H), 4.94 (s, 2 H), 3.61 (dd, 1H), 2.87 (d, 1H), 2.60 (d, 1H), 2.52-2.55 (t, 1H), 2.20 (q, 2H), 1.86-2.05 (m, 3 H), 1.74-1.84 (m, 1H), 1.71 (s, 3H), 1.62 (s, 3H), 1.04-1.17 (m, 4H).

### Example 9: (3R,4S,5S,6R)-5-methoxy-4-((2R,3R)-2-methyl-3-(3-methylbut-2-en-1-yl)oxiran-2-yl)-1-oxaspiro[2.5]octan-6-yl (4-nitrophenyl) carbonate (Intermediate 8)

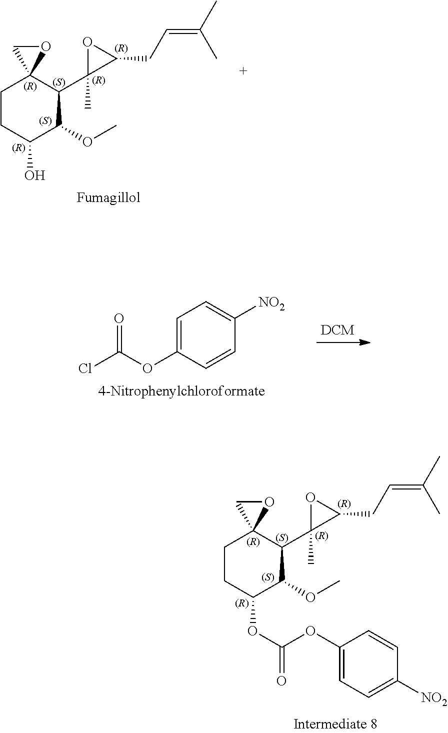

(3R,4S,5S,6R)-5-methoxy-4-((2R,3R)-2-methyl-3-(3-methylbut-2-en-1-yl)oxiran-2-yl)-1-oxaspiro[2.5]octan-6-ol (fumagillol) (3.4 g, 12.0 mmol) was dissolved in 50 ml of dimethylformamide (DMF) and diispropylethyamine (3.5 mL) was added. The reaction mixture was stirred at 0° C. in an ice bath for 1 hour. 4-nitrophenyl chloroformate (2.9 g, 14.4 mmol) was added to this stirred solution at 0° C. After addition, the reaction mixture was stirred at 0° C. and stirred for 6 hours and then at room temperature for 12 hours. The reaction was monitored by TLC. After the fumagillol starting material was completely consumed, the reaction mixture was diluted with 50 mL of dichloromethane and washed with brine solution water and water (50 mL×3). The organic layers were combined and dried over anhydrous Na₂SO₄. After filtration, the filtrate was concentrated under reduced pressure to afford 3.2 g of the intermediate 8 as a yellow oil.

### Example 10: (4-(((((3R,4S,5S,6R)-5-methoxy-4-((2R,3R)-2-methyl-3-(3-methylbut-2-en-1-yl)oxiran-2-yl)-1-oxaspiro[2.5]octan-6-yl)oxy)carbonyl)amino)phenyl)boronic acid (Table A, No. 17)

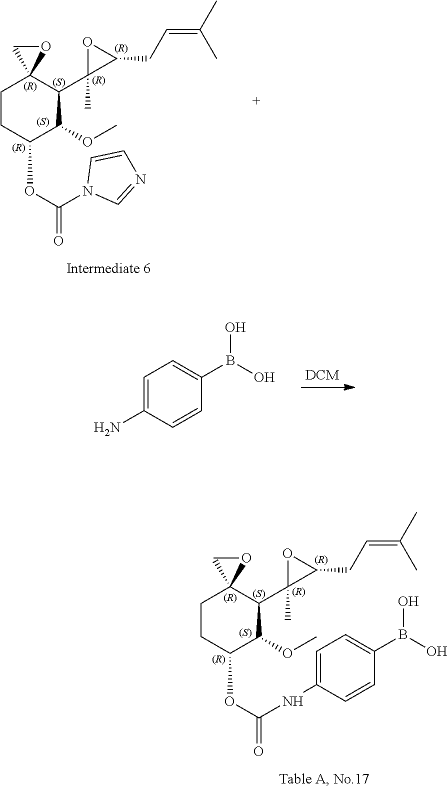

The intermediate 6 (376 mg, 1 mmol) was dissolved in 5 mL of dichloromethane. To this solution was added a mixture of (4-aminophenyl)boronic acid (411 mg, 3 mmol) and N,N-Diisopropylethylamine (387 mg, 3 mmol) in 5 mL of dichloromethane was added. The reaction mixture was stirred at 0° C. Next, a solution of the key intermediate 1 (376 mg, 1 mmol) in 5 mL of dichloromethane was added to this reaction mixture. The reaction mixture was stirred for 16 hours at room temparature. The reaction was monitored by TLC. After the key intermediate 1 was completely consumed, the reaction mixture was concentrated. It was then diluted with 20 ml of dichloromethane and washed with ammonium acetate buffer (pH 4.0, 5 mL x2). The dichloromethane layer was collected and dried over anhydrous Na₂SO₄. After filtration, the filtrate was concentrated to give a crude product, which was purified by preparative HPLC to give the title compound as a semi-solid (210 mg).

### Example 11: (4-(((((3R,4S,5S,6R)-5-methoxy-4-((2R,3R)-2-methyl-3-(3-methylbut-2-en-1-yl)oxiran-2-yl)-1-oxaspiro[2.5]octan-6-yl)oxy)carbonyl)amino)phenyl)boronic acid (Table A, No. 17)

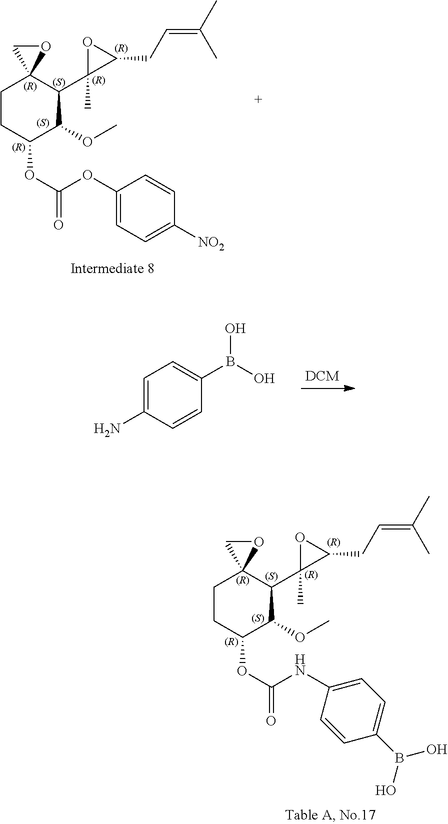

The intermediate 8 (448, 1 mmol) was dissolved in 5 mL of DMF. To this solution was added a mixture of (4-aminophenyl)boronic acid (417 mg, 3 mmol) and N,N-Diisopropylethylamine (323 mg, 2.5 mmol) in 5 mL of DMF was added. The reaction mixture was stirred at 0° C. A solution of the key intermediate 2 (447 mg, 1 mmol) in 5 mL of DMF was added to this reaction mixture. The reaction mixture was stirred for 16 hours at room temperature. After the key intermediate 1 was completely consumed, the reaction mixture was concentrated. It was then diluted with 20 ml of ethyl acetate. The organic layer was washed twice with ammonium acetate buffer (pH 4.0, 5 mL). The organic layer was collected and dried over anhydrous Na₂SO₄. After filtration, the filtrate was concentrated to give the crude product, which was purified by preparative HPLC to give the title compound as a semi-solid (96 mg).

### Biological Assays

### Trophozoite Cultures

Giardia cultures: Trophozoites of G. lamblia isolates WB and GS were grown anaerobically in borosilicate glass screw-cap culture tubes (Fisher Scientific) at pH 7.0 in modified TYI-S-33 medium. The medium was supplemented with 10% heat-inactivated bovine serum (Sigma-Aldrich) and 0.05% bovine bile (Sigma-Aldrich). To attain low-oxygen-tension conditions, the tubes were filled to 85 to 90% of their total volume capacity and incubated without shaking at 37° C. Subcultures (2×105 trophozoites per tube) were made three times a week. Detachment of trophozoites for inoculation was achieved by chilling the cultures on ice for 20 min. Culturing and detachment of Metronidazole-resistant G. lamblia Assemblage A 713M3 and assemblage B 1279-M1 trophozoites followed the same protocol, except that the growth medium was supplemented with metronidazole gradually increasing the concentration to 10 μM. Amebae cultures: E. histolytica strain HM1:IMSS trophozoites were grown at 37° C. in TYI-S-33 medium supplemented with penicillin (100 U/mL) and streptomycin sulfate (100 μg/mL). Trophozoites were grown anaerobically in borosilicate tubes and subcultures were made 1-2 times a week. Trophozoites were detached for inoculation by chilling the cultures on ice for 20 minutes.

Trophozoite viability IC₅₀determination

Giardia assays: 10 μL giardia trophozoites in growth medium were plated at a density of 10,000 cells/well in sterile 96-well black clear bottom assay plates. Fumagillin and fumagillol derivatives were 1:3 serially diluted from a 1 μM DMSO stock solution and then 100 μL/well were transferred in duplicate to the assay wells. Metronidazole (control) was 1:3 serially diluted in growth medium from a 100 μM stock solution in DMSO. The assay plates were placed in a BD GasPak™ EZ Container System (BD Diagnostics) to create an anaerobic growth environment. The sealed containers were incubated at 37° C. for 72 hr. Following incubation, 70 μL/well of the ATPLite reagent (PerkinElmer) was added to the assay plates for one-step lysis and ATP level detection. The luminescent signals of the assay plates were measured on an EnSpire 2300 plate reader (PerkinElmer). Concentration response titration points for each compound were fitted to a 4-parameters logistic nonlinear regression model using KaleidaGraph, yielding the IC₅₀value and standard error (SE).

Amebae assays: The protocol is identical to that described above except that the trophozoites were plated at a density of 5000 cells/well because of their larger size.

### MetAP2 Fluorescence Assay

Recombinant human MetAP2 (Bio-Techne/R&D systems) was diluted to 10 μg/mL in the assay buffer (50 mM HEPES, 0.1 mM CoCl2, 100 mM NaCl, pH 7.5). The substrate, H-Met-Gly-Pro-AMC (Bio-Techne/R&D systems), was diluted to 500 μM with 2 μg/mL of the coupling enzyme, recombinant human dipeptidyl peptidase-4 (Bio-Techne/R&D systems), in assay buffer. 50 μL of 10 μg/mL recombinant human METAP2 were loaded into a flat-bottom black 96-well plate (Greiner Bio), and the reaction was initiated by adding 50 μL of the substrate/dipeptidyl peptidase-4 mixture. The reaction mixture was incubated at room temperature for 10 minutes, and the fluorescence was measured for 5 minutes at excitation and emission wavelengths of 380 nm and 460 nm, respectively, in kinetic mode. For IC₅₀determination, 0.5 μL inhibitor solution was added to the reaction mixture. The reaction rates were used to calculate IC₅₀with KaleidaGraph.

TABLE A

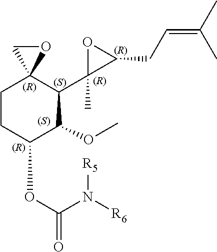

| | | | | Cell viability values Mean | | |
|---|---|---|---|---|---|---|
| | | | | IC₅₀ (nM) | | |
| No | 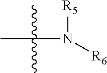 | Molecular Weight | MetAP2 IC₅₀ (nM) | G. lamblia GS | G. lamblia WB | E. histolytica HM1: IMSS |
| 1 | 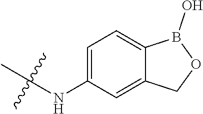 [SMILES: *Nc1ccc2c(c1)COB2O] | 457.33 | + | + | + | + |
| 2 | 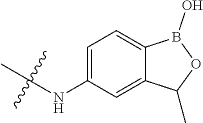 [SMILES: *Nc1ccc2c(c1)C(C)OB2O] | 471.357 | + | + | + | + |
| 3 | 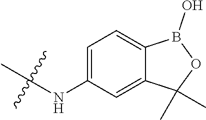 [SMILES: *Nc1ccc2c(c1)C(C)(C)OB2O] | 485.384 | + | + | + | + |
| 4 | 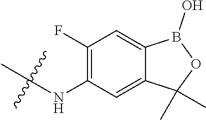 [SMILES: *Nc1cc2c(cc1F)B(O)OC2(C)C] | 503.3744 | + | + | ++ | ++ |
| 5 | 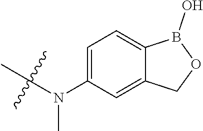 [SMILES: *N(C)c1ccc2c(c1)COB2O] | 471.357 | + | ++ | ++ | ++ |
| 6 | 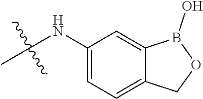 [SMILES: *Nc1ccc2c(c1)B(O)OC2] | 457.33 | + | + | ++ | ++ |
| 7 | 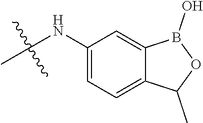 [SMILES: *Nc1ccc2c(c1)B(O)OC2C] | 471.357 | + | + | ++ | ++ |
| 8 | 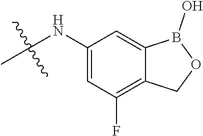 [SMILES: *Nc1cc(F)c2c(c1)B(O)OC2] | 475.3204 | + | + | ++ | ++ |
| 9 | 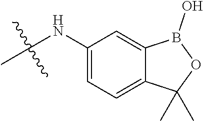 [SMILES: *Nc1ccc2c(c1)B(O)OC2(C)C] | 485.384 | + | + | ++ | ++ |
| 10 | 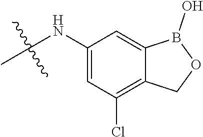 [SMILES: *Nc1cc(Cl)c2c(c1)B(O)OC2] | 491.772 | + | ++ | ++ | ++ |
| 11 | 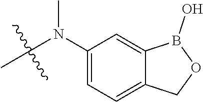 [SMILES: *N(C)c1ccc2c(c1)B(O)OC2] | 471.357 | ++ | ++ | +++ | +++ |
| 12 | 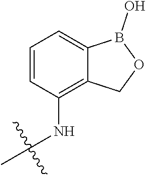 [SMILES: *Nc1cccc2c1COB2O] | 457.33 | + | ++ | +++ | +++ |
| 13 | 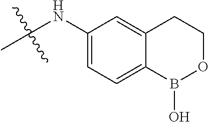 [SMILES: *Nc1ccc2c(c1)CCOB2O] | 471.357 | + | + | ++ | +++ |
| 14 | 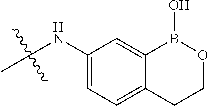 [SMILES: *Nc1ccc2c(c1)B(O)OCC2] | 471.357 | + | ++ | +++ | +++ |
| 15 | 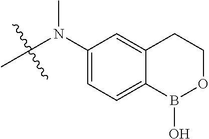 [SMILES: *N(C)c1ccc2c(c1)CCOB2O] | 485.384 | + | +++ | +++ | +++ |
| 16 | 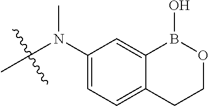 [SMILES: *N(C)c1ccc2c(c1)B(O)OCC2] | 485.384 | + | +++ | +++ | +++ |
| 17 | 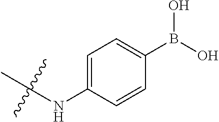 [SMILES: *Nc1ccc(B(O)O)cc1] | 445.319 | + | + | ++ | ++ |
| 18 | 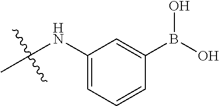 [SMILES: *Nc1cccc(B(O)O)c1] | 445.319 | + | ++ | +++ | +++ |
| 19 | 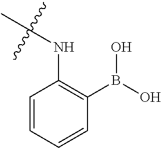 [SMILES: *Nc1ccccc1B(O)O] | 445.319 | + | ++ | +++ | +++ |
| 20 | 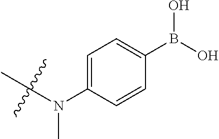 [SMILES: *N(C)c1ccc(B(O)O)cc1] | 459.346 | + | +++ | +++ | +++ |
| 21 | 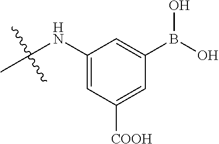 [SMILES: *Nc1cc(B(O)O)cc(C(=O)O)c1] | 489.328 | + | +++ | +++ | +++ |
| 22 | 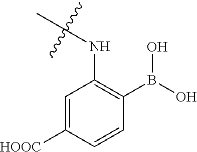 [SMILES: *Nc1cc(C(=O)O)ccc1B(O)O] | 489.328 | + | +++ | +++ | +++ |
| 23 | 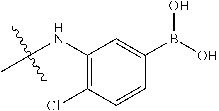 [SMILES: *Nc1cc(B(O)O)ccc1Cl] | 479.761 | + | +++ | +++ | +++ |
| 24 | 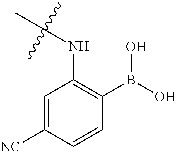 [SMILES: *Nc1cc(C#N)ccc1B(O)O] | 470.329 | + | +++ | +++ | ++++ |
| 25 | 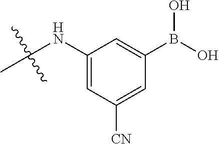 [SMILES: *Nc1cc(C#N)cc(B(O)O)c1] | 470.329 | + | +++ | +++ | ++++ |
| 26 | 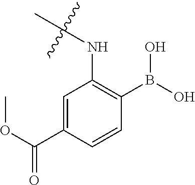 [SMILES: *Nc1cc(C(=O)OC)ccc1B(O)O] | 503.355 | ++ | +++ | ++++ | ++++ |
| 27 | 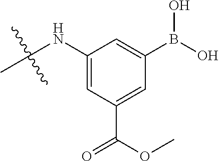 [SMILES: *Nc1cc(B(O)O)cc(C(=O)OC)c1] | 503.355 | +++ | +++ | +++ | +++ |
| 28 | 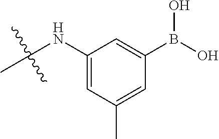 [SMILES: *Nc1cc(C)cc(B(O)O)c1] | 459.346 | + | ++ | +++ | +++ |
| 29 | 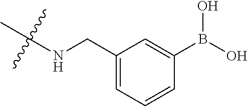 [SMILES: *NCc1cccc(B(O)O)c1] | 459.346 | + | +++ | +++ | +++ |
| 30 | 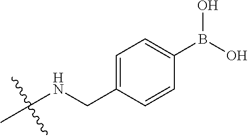 [SMILES: *NCc1ccc(B(O)O)cc1] | 459.346 | + | +++ | +++ | +++ |
| 31 | 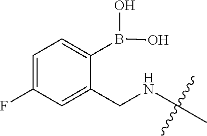 [SMILES: *NCc1cc(F)ccc1B(O)O] | 477.3364 | + | +++ | +++ | ++++ |
| 32 | 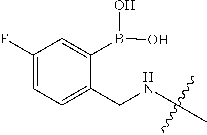 [SMILES: *NCc1ccc(F)cc1B(O)O] | 477.3364 | ++ | +++ | ++++ | ++++ |
| 33 | 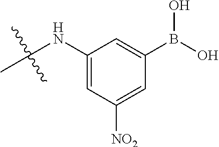 [SMILES: *Nc1cc(B(O)O)cc([N+](=O)[O-])c1] | 490.316 | + | +++ | ++++ | ++++ |
| 34 | 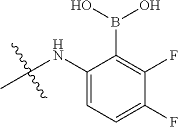 [SMILES: *Nc1ccc(F)c(F)c1B(O)O] | 481.2998 | + | +++ | ++++ | ++++ |
| 35 | 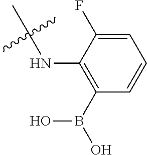 [SMILES: *Nc1c(F)cccc1B(O)O] | 463.3094 | + | +++ | ++++ | ++++ |
| 36 | 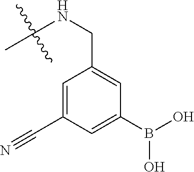 [SMILES: *NCc1cc(C#N)cc(B(O)O)c1] | 484.356 | ++ | +++ | ++++ | ++++ |
| 37 | 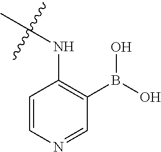 [SMILES: *Nc1ccncc1B(O)O] | 446.307 | + | ++ | ++++ | ++++ |
| 38 | 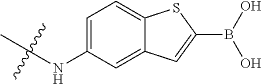 [SMILES: *Nc1ccc2sc(B(O)O)cc2c1] | 501.401 | + | ++++ | ++++ | ++++ |
| 39 | 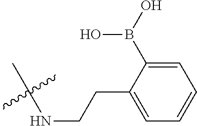 [SMILES: *NCCc1ccccc1B(O)O] | 473.373 | + | +++ | ++++ | +++++ |
| 40 | 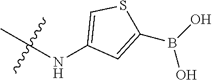 [SMILES: *Nc1csc(B(O)O)c1] | 451.341 | + | +++ | ++++ | ++++ |
| 41 | 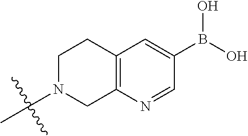 [SMILES: *N1CCc2cc(B(O)O)cnc2C1] | 486.372 | + | ++ | +++ | ++++ |
| 42 | 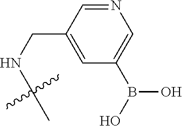 [SMILES: *NCc1cncc(B(O)O)c1] | 460.334 | ++ | +++ | ++++ | ++++ |
| 43 | 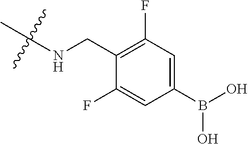 [SMILES: *NCc1c(F)cc(B(O)O)cc1F] | 495.3268 | + | +++ | ++++ | ++++ |
| 44 | 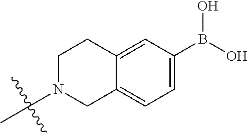 [SMILES: *N1CCc2cc(B(O)O)ccc2C1] | 485.384 | + | ++ | ++++ | ++++ |
| 45 | 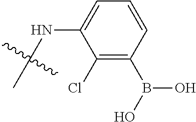 [SMILES: *Nc1cccc(B(O)O)c1Cl] | 479.761 | + | +++ | ++++ | ++++ |
| 46 | 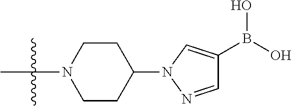 [SMILES: *N1CCC(n2cc(B(O)O)cn2)CC1] | 503.403 | ++ | +++ | ++++ | ++++ |
| 47 | 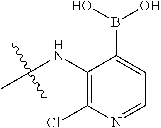 [SMILES: *Nc1c(B(O)O)ccnc1Cl] | 480.749 | + | +++ | ++++ | ++++ |
| 48 | 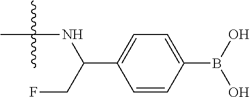 [SMILES: *NC(CF)c1ccc(B(O)O)cc1] | 491.3634 | + | +++ | ++++ | ++++ |
| 49 | 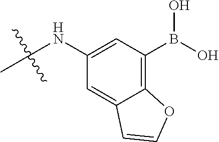 [SMILES: *Nc1cc(B(O)O)c2occc2c1] | 485.34 | + | ++ | +++ | ++++ |
| 50 | 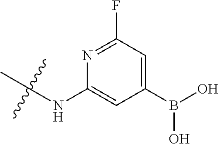 [SMILES: *Nc1cc(B(O)O)cc(F)n1] | 464.2974 | + | ++ | ++++ | ++++ |
| 51 | 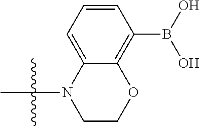 [SMILES: *N1CCOc2c(B(O)O)cccc21] | 487.356 | + | + | +++ | +++ |
| 52 | 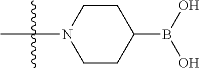 [SMILES: *N1CCC(B(O)O)CC1] | 437.34 | + | ++ | +++ | ++++ |
| 53 | 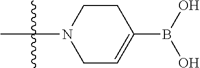 [SMILES: *N1CC=C(B(O)O)CC1] | 435.324 | + | +++ | ++++ | ++++ |
| 54 | 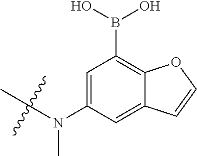 [SMILES: *N(C)c1cc(B(O)O)c2occc2c1] | 499.367 | + | +++ | ++++ | ++++ |
| 55 | 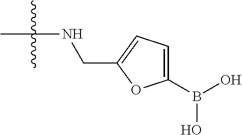 [SMILES: *NCc1ccc(B(O)O)o1] | 449.307 | + | +++ | ++++ | ++++ |
| 56 | 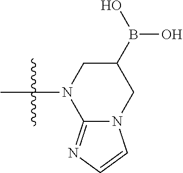 [SMILES: *N1CC(B(O)O)Cn2ccnc21] | 475.349 | ++ | +++ | ++++ | ++++ |
| 57 | 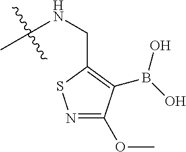 [SMILES: *NCc1snc(OC)c1B(O)O] | 496.382 | ++ | +++ | ++++ | ++++ |
| 58 | 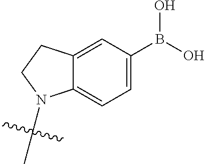 [SMILES: *N1CCc2cc(B(O)O)ccc21] | 471.357 | + | +++ | ++++ | ++++ |
| 59 | 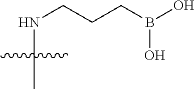 [SMILES: *NCCCB(O)O] | 411.302 | + | ++++ | ++++ | ++++ |
| 60 | 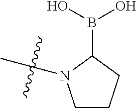 [SMILES: *N1CCCC1B(O)O] | 423.313 | + | ++ | ++++ | ++++ |
| 61 | 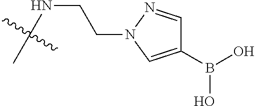 [SMILES: *NCCn1cc(B(O)O)cn1] | 463.338 | ++ | +++ | ++++ | ++++ |
| 62 | 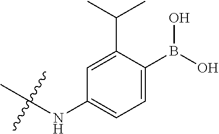 [SMILES: *Nc1ccc(B(O)O)c(C(C)C)c1] | 487.4 | + | +++ | ++++ | ++++ |
| 63 | 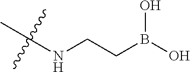 [SMILES: *NCCB(O)O] | 397.275 | + | ++ | +++ | +++ |
| 64 | 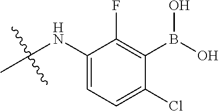 [SMILES: *Nc1ccc(Cl)c(B(O)O)c1F] | 497.7514 | + | +++ | ++++ | ++++ |
| 65 | 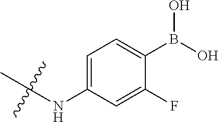 [SMILES: *Nc1ccc(B(O)O)c(F)c1] | 463.3 | + | + | +++ | ++++ |
| 66 | 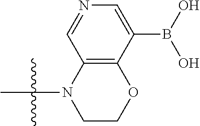 [SMILES: *N1CCOc2c(B(O)O)cncc21] | 488.344 | ++ | +++ | ++++ | ++++ |
| 67 | 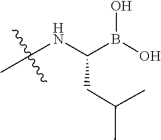 [SMILES: *N[C@@H](CC(C)C)B(O)O] | 439.356 | + | ++ | +++ | +++ |
| 68 | 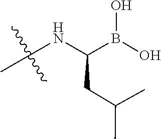 [SMILES: *N[C@H](CC(C)C)B(O)O] | 439.356 | + | +++ | ++++ | ++++ |
| 69 | 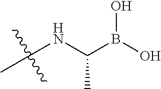 [SMILES: *N[C@@H](C)B(O)O] | 397.275 | + | ++ | ++++ | ++++ |
| 70 | 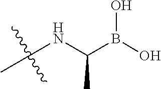 [SMILES: *N[C@H](C)B(O)O] | 397.275 | + | ++ | +++ | ++++ |

IC₅₀ Values:
<10 nM (+), 10.1-100 nM (++), 101-1000 nM (+++) and >1000 nM (++++)

TABLE B

| No | IUPAC Chemical Name |
|---|---|
| 1 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-1,3- |
| | dihydrobenzo[c][1,2]oxaborol-5-yl)carbamate |
| 2 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-3-methyl-1,3- |
| | dihydrobenzo[c][1,2]oxaborol-5-yl)carbamate |
| 3 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-3,3-dimethyl-1,3- |
| | dihydrobenzo[c][1,2]oxaborol-5-yl)carbamate |
| 4 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (6-fluoro-1-hydroxy-3,3-dimethyl-1,3- |
| | dihydrobenzo[c][1,2]oxaborol-5-yl)carbamate |
| 5 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-1,3- |
| | dihydrobenzo[c][1,2]oxaborol-5-yl)(methyl)carbamate |
| 6 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-1,3- |
| | dihydrobenzo[c][1,2]oxaborol-6-yl)carbamate |
| 7 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-3-methyl-1,3- |
| | dihydrobenzo[c][1,2]oxaborol-6-yl)carbamate |
| 8 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (4-fluoro-1-hydroxy-1,3- |
| | dihydrobenzo[c][1,2]oxaborol-6-yl)carbamate |
| 9 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-3,3-dimethyl-1,3- |
| | dihydrobenzo[c][1,2]oxaborol-6-yl)carbamate |
| 10 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (4-chloro-1-hydroxy-1,3- |
| | dihydrobenzo[c][1,2]oxaborol-6-yl)carbamate |
| 11 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-1,3- |
| | dihydrobenzo[c][1,2]oxaborol-6-yl)(methyl)carbamate |
| 12 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-1,3- |
| | dihydrobenzo[c][1,2]oxaborol-4-yl)carbamate |
| 13 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-3,4-dihydro-1H- |
| | benzo[c][1,2]oxaborinin-6-yl)carbamate |
| 14 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-3,4-dihydro-1H- |
| | benzo[c][1,2]oxaborinin-7-yl)carbamate |
| 15 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-3,4-dihydro-1H- |
| | benzo[c][1,2]oxaborinin-6-yl)(methyl)carbamate |
| 16 | (3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl (1-hydroxy-3,4-dihydro-1H- |
| | benzo[c][1,2]oxaborinin-7-yl)(methyl)carbamate |
| 17 | (4-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)phenyl)boronic |
| | acid |
| 18 | (3-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)phenyl)boronic |
| | acid |
| 19 | (2-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)phenyl)boronic |
| | acid |
| 20 | (4-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6- |
| | yl)oxy)carbonyl)(methyl)amino)phenyl)boronic acid |
| 21 | 3-borono-5-(((((3R,4S,5S,6R)-5-methoxy-4-((2R,3R)-2-methyl-3-(3-methylbut- |
| | 2-en-1-yl)oxiran-2-yl)-1-oxaspiro[2.5]octan-6-yl)oxy)carbonyl)amino)benzoic |
| | acid |
| 22 | 4-borono-3-(((((3R,4S,5S,6R)-5-methoxy-4-((2R,3R)-2-methyl-3-(3-methylbut- |
| | 2-en-1-yl)oxiran-2-yl)-1-oxaspiro[2.5]octan-6-yl)oxy)carbonyl)amino)benzoic |
| | acid |
| 23 | (4-chloro-3-(((((3R,4S,5S,6R)-5-methoxy-4-((2R,3R)-2-methyl-3-(3-methylbut- |
| | 2-en-1-yl)oxiran-2-yl)-1-oxaspiro[2.5]octan-6- |
| | yl)oxy)carbonyl)amino)phenyl)boronic acid |
| 24 | (4-cyano-2-(((((3R,4S,5S,6R)-5-methoxy-4-((2R,3R)-2-methyl-3-(3-methylbut- |
| | 2-en-1-yl)oxiran-2-yl)-1-oxaspiro[2.5]octan-6- |
| | yl)oxy)carbonyl)amino)phenyl)boronic acid |
| 25 | (3-cyano-5-(((((3R,4S,5S,6R)-5-methoxy-4-((2R,3R)-2-methyl-3-(3-methylbut- |
| | 2-en-1-yl)oxiran-2-yl)-1-oxaspiro[2.5]octan-6- |
| | yl)oxy)carbonyl)amino)phenyl)boronic acid |
| 26 | (2-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)-4- |
| | (methoxycarbonyl)phenyl)boronic acid |
| 27 | (3-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)-5- |
| | (methoxycarbonyl)phenyl)boronic acid |
| 28 | (3-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)-5- |
| | methylphenyl)boronic acid |
| 29 | (3-((((((3R,4S,5S,6R)-5-methoxy-4-((2R,3R)-2-methyl-3-(3-methylbut-2-en-1- |
| | yl)oxiran-2-yl)-1-oxaspiro[2.5]octan-6- |
| | yl)oxy)carbonyl)amino)methyl)phenyl)boronic acid |
| 30 | (4-((((((3R,4S,5S,6R)-5-methoxy-4-((2R,3R)-2-methyl-3-(3-methylbut-2-en-1- |
| | yl)oxiran-2-yl)-1-oxaspiro[2.5]octan-6- |
| | yl)oxy)carbonyl)amino)methyl)phenyl)boronic acid |
| 31 | (4-fluoro-2-((((((3R,4S,5S,6R)-5-methoxy-4-((2R,3R)-2-methyl-3-(3-methylbut- |
| | 2-en-1-yl)oxiran-2-yl)-1-oxaspiro[2.5]octan-6- |
| | yl)oxy)carbonyl)amino)methyl)phenyl)boronic acid |
| 32 | (5-fluoro-2-((((((3R,4S,5S,6R)-5-methoxy-4-((2R,3R)-2-methyl-3-(3-methylbut- |
| | 2-en-1-yl)oxiran-2-yl)-1-oxaspiro[2.5]octan-6- |
| | yl)oxy)carbonyl)amino)methyl)phenyl)boronic acid |
| 33 | (3-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)-5- |
| | nitrophenyl)boronic acid |
| 34 | (2,3-difluoro-6-(((((3R,4S,5S,6R)-5-methoxy-4-((2R,3R)-2-methyl-3-(3- |
| | methylbut-2-en-1-yl)oxiran-2-yl)-1-oxaspiro[2.5]octan-6- |
| | yl)oxy)carbonyl)amino)phenyl)boronic acid |
| 35 | (3-fluoro-2-(((((3R,4S,5S,6R)-5-methoxy-4-((2R,3R)-2-methyl-3-(3-methylbut-2- |
| | en-1-yl)oxiran-2-yl)-1-oxaspiro[2.5]octan-6- |
| | yl)oxy)carbonyl)amino)phenyl)boronic acid |
| 36 | (3-cyano-5-((((((3R,4S,5S,6R)-5-methoxy-4-((2R,3R)-2-methyl-3-(3-methylbut- |
| | 2-en-1-yl)oxiran-2-yl)-1-oxaspiro[2.5]octan-6- |
| | yl)oxy)carbonyl)amino)methyl)phenyl)boronic acid |
| 37 | (4-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)pyridin-3- |
| | yl)boronic acid |
| 38 | (5-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)benzo[b]thiophen- |
| | 2-yl)boronic acid |
| 39 | (2-(2-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6- |
| | yl)oxy)carbonyl)amino)ethyl)phenyl)boronic acid |
| 40 | (4-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)thiophen-2- |
| | yl)boronic acid |
| 41 | (7-((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)-5,6,7,8-tetrahydro-1,7- |
| | naphthyridin-3-yl)boronic acid |
| 42 | (5-((((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)methyl)pyridin-3- |
| | yl)boronic acid |
| 43 | (3,5-difluoro-4-((((((3R,4S,5S,6R)-5-methoxy-4-((2R,3R)-2-methyl-3-(3- |
| | methylbut-2-en-1-yl)oxiran-2-yl)-1-oxaspiro[2.5]octan-6- |
| | yl)oxy)carbonyl)amino)methyl)phenyl)boronic acid |
| 44 | (2-((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)-1,2,3,4- |
| | tetrahydroisoquinolin-6-yl)boronic acid |
| 45 | (2-chloro-3-(((((3R,4S,5S,6R)-5-methoxy-4-((2R,3R)-2-methyl-3-(3-methylbut- |
| | 2-en-1-yl)oxiran-2-yl)-1-oxaspiro[2.5]octan-6- |
| | yl)oxy)carbonyl)amino)phenyl)boronic acid |
| 46 | (1-(1-((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)piperidin-4-yl)-1H- |
| | pyrazol-4-yl)boronic acid |
| 47 | (2-chloro-3-(((((3R,4S,5S,6R)-5-methoxy-4-((2R,3R)-2-methyl-3-(3-methylbut- |
| | 2-en-1-yl)oxiran-2-yl)-1-oxaspiro[2.5]octan-6-yl)oxy)carbonyl)amino)pyridin-4- |
| | yl)boronic acid |
| 48 | (4-(2-fluoro-1-(((((3R,4S,5S,6R)-5-methoxy-4-((2R,3R)-2-methyl-3-(3- |
| | methylbut-2-en-1-yl)oxiran-2-yl)-1-oxaspiro[2.5]octan-6- |
| | yl)oxy)carbonyl)amino)ethyl)phenyl)boronic acid |
| 49 | (5-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)benzofuran-7- |
| | yl)boronic acid |
| 50 | (2-fluoro-6-(((((3R,4S,5S,6R)-5-methoxy-4-((2R,3R)-2-methyl-3-(3-methylbut-2- |
| | en-1-yl)oxiran-2-yl)-1-oxaspiro[2.5]octan-6-yl)oxy)carbonyl)amino)pyridin-4- |
| | yl)boronic acid |
| 51 | (4-((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)-3,4-dihydro-2H- |
| | benzo[b][1,4]oxazin-8-yl)boronic acid |
| 52 | (1-((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)piperidin-4-yl)boronic |
| | acid |
| 53 | (1-((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)-1,2,3,6- |
| | tetrahydropyridin-4-yl)boronic acid |
| 54 | (5-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6- |
| | yl)oxy)carbonyl)(methyl)amino)benzofuran-7-yl)boronic acid |
| 55 | (5-((((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)methyl)furan-2- |
| | yl)boronic acid |
| 56 | (8-((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)-5,6,7,8- |
| | tetrahydroimidazo[1,2-a]pyrimidin-6-yl)boronic acid |
| 57 | (3-methoxy-5-((((((3R,4S,5S,6R)-5-methoxy-4-((2R,3R)-2-methyl-3-(3- |
| | methylbut-2-en-1-yl)oxiran-2-yl)-1-oxaspiro[2.5]octan-6- |
| | yl)oxy)carbonyl)amino)methyl)isothiazol-4-yl)boronic acid |
| 58 | (1-((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)indolin-5-yl)boronic acid |
| 59 | (3-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)propyl)boronic |
| | acid |
| 60 | (1-((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)pyrrolidin-2-yl)boronic |
| | acid |
| 61 | (1-(2-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)ethyl)-1H-pyrazol- |
| | 4-yl)boronic acid |
| 62 | (2-isopropyl-4-(((((3R,4S,5S,6R)-5-methoxy-4-((2R,3R)-2-methyl-3-(3- |
| | methylbut-2-en-1-yl)oxiran-2-yl)-1-oxaspiro[2.5]octan-6- |
| | yl)oxy)carbonyl)amino)phenyl)boronic acid |
| 63 | (2-(((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)amino)ethyl)boronic acid |
| 64 | (6-chloro-2-fluoro-3-(((((3R,4S,5S,6R)-5-methoxy-4-((2R,3R)-2-methyl-3-(3- |
| | methylbut-2-en-1-yl)oxiran-2-yl)-1-oxaspiro[2.5]octan-6- |
| | yl)oxy)carbonyl)amino)phenyl)boronic acid |
| 65 | (2-fluoro-4-(((((3R,4S,5S,6R)-5-methoxy-4-((2R,3R)-2-methyl-3-(3-methylbut-2- |
| | en-1-yl)oxiran-2-yl)-1-oxaspiro[2.5]octan-6- |
| | yl)oxy)carbonyl)amino)phenyl)boronic acid |
| 66 | (4-((((3R,4S,5S,6R)-5-methoxy-4-((2R,3R)-2-methyl-3-(3-methylbut-2-en-1- |
| | yl)oxiran-2-yl)-1-oxaspiro[2.5]octan-6-yl)oxy)carbonyl)-3,4-dihydro-2H- |
| | pyrido[4,3-b][1,4]oxazin-8-yl)boronic acid |
| 67 | ((R)-1-(((((3R,4S,5S,6R)-5-methoxy-4-((2R,3R)-2-methyl-3-(3-methylbut-2-en- |
| | 1-yl)oxiran-2-yl)-1-oxaspiro[2.5]octan-6-yl)oxy)carbonyl)amino)-3- |
| | methylbutyl)boronic acid |
| 68 | ((S)-1-(((((3R,4S,5S,6R)-5-methoxy-4-((2R,3R)-2-methyl-3-(3-methylbut-2-en- |
| | 1-yl)oxiran-2-yl)-1-oxaspiro[2.5]octan-6-yl)oxy)carbonyl)amino)-3- |
| | methylbutyl)boronic acid |
| 69 | ((R)-1-(((((3R,4S,5S,6R)-5-methoxy-4-((2R,3R)-2-methyl-3-(3-methylbut-2-en- |
| | 1-yl)oxiran-2-yl)-1-oxaspiro[2.5]octan-6-yl)oxy)carbonyl)amino)ethyl)boronic |
| | acid |
| 70 | ((S)-1-(((((3R,4S,5S,6R)-5-methoxy-4-((2R,3R)-2-methyl-3-(3-methylbut-2-en- |
| | 1-yl)oxiran-2-yl)-1-oxaspiro[2.5]octan-6-yl)oxy)carbonyl)amino)ethyl)boronic |
| | acid |

### REFERENCES

Agner, G., Gyorbiro, A., Valko, I., Vasvari-Debreczy, L., Szabo, A., Farkas, G., . . . Hermecz, I. (2003). [Light- and temperature-induced degradation of fumagillin]. Acta Pharm Hung, 73(1), 41-45. http://ww.ncbi.nlm.nih.gov/pubmed/12891898 (Fumagillin ho es feny hatasara torteno bomlasanak vizsgalata.)
Arico-Muendel, C., Centrella, P. A., Contonio, B. D., Morgan, B. A., O'Donovan, G., Paradise, C. L., . . . Chiang, P. K. (2009). Antiparasitic activities of novel, orally available fumagillin analogs. Bioorg Med Chem Lett, 19(17), 5128-5131. https://doi.org/10.1016/j.bmel.2009.07.029
Arico-Muendel, C. C., Benjamin, D. R., Caiazzo, T. M., Centrella, P. A., Contonio, B. D., Cook, C. M., . . . White, K. F. (2009). Carbamate analogues of fumagillin as potent, targeted inhibitors of methionine aminopeptidase-2. J Med Chem, 52(24), 8047-8056. https://doi.org/10.1021/jm90126k
Baker, S. J., Ding, C. Z., Akama, T., Zhang, Y.-K., Hernandez, V., & Xia, Y. (2009). Therapeutic potential of boron-containing compounds. Future medicinal chemistry, 1(7), 1275-1288.
Ban, H. S., & Nakamura, H. (2015). Boron-based drug design. The Chemical Record, 15(3), 616-635.
Burkey, B. F., Hoglen, N. C., Inskeep, P., Wyman, M., Hughes, T. E., & Vath, J. E. (2018). Preclinical efficacy and safety of the novel antidiabetic, antiobesity MetAP2 inhibitor ZGN-1061. Journal of Pharmacology and Experimental Therapeutics, 365(2), 301-313.
Champion, L., Durrbach, A., Lang, P., Delahousse, M., Chauvet, C., Sarfati, C., . . . Molina, J. M. (2010). Fumagillin for treatment of intestinal microsporidiosis in renal transplant recipients [Case Reports]. American journal of transplantation: official journal of the American Society of Transplantation and the American Society of Transplant Surgeons, 10(8), 1925-1930. https://doi.org/10.111/i.1600-6143.2010.03166.x
Chen, X., Xie, S., Bhat, S., Kumar, N., Shapiro, T. A., & Liu, J. O. (2009). Fumagillin and fumarranol interact with P. falciparum methionine aminopeptidase 2 and inhibit malaria parasite growth in vitro and in vivo [Research Support, N.I.H., Extramural
Research Support, Non-U.S. Gov't]. Chemistry & biology, 16(2), 193-202.
Chong, P. Y., Shotwell, J. B., Miller, J., Price, D. J., Maynard, A., Voitenleitner, C., . . . Peat, A. J. (2019). Design of N-Benzoxaborole Benzofuran GSK8175-Optimization of Human Pharmacokinetics Inspired by Metabolites of a Failed Clinical HCV Inhibitor. J Med Chem, 62(7), 3254-3267. https://doi.org/10.1021/acs.jmedchem.8b01719
Conteas, C., Berlin, O., Ash, L., & Pruthi, J. (2000). Therapy for human gastrointestinal microsporidiosis. The American journal of tropical medicine and hygiene, 63(3), 121-127.
Demers, M., Sturtevant, S., Guertin, K. R., Gupta, D., Desai, K., Vieira, B. F., . . . Gongalves, B. P. (2021). MetAP2 inhibition modifies hemoglobin S to delay polymerization and improves blood flow in sickle cell disease. Blood advances, 5(5), 1388-1402.
Didier, E. S. (1997). Effects of albendazole, fumagillin, and TNP-470 on microsporidial replication in vitro. Antimicrobial agents and chemotherapy, 41(7), 1541-1546.
Didier, P. J., Phillips, J. N., Kuebler, D. J., Nasr, M., Brindley, P. J., Stovall, M. E., . . . Didier, E. (2006). Antimicrosporidial activities of fumagillin, TNP-470, ovalicin, and ovalicin derivatives in vitro and in vivo. Antimicrobial agents and chemotherapy, 50(6), 2146-2155.
Diesenhouse, M. C., Wilson, L. A., Corrent, G. F., Visvesvara, G. S., Grossniklaus, H. E., & Bryan, R. T. (1993). Treatment of microsporidial keratoconjunctivitis with topical fumagillin [Case Reports
Research Support, Non-U.S. Gov't
Research Support, U.S. Gov't, P.H.S.]. American journal of ophthalmology, 115(3), 293-298. http://ww.ncbi.nlm.nih.gov/pubmed/8117342
Freund, Y. R., Akama, T., Alley, M., Antunes, J., Dong, C., Jarnagin, K., . . . Plattner, J. J. (2012). Boron-based phosphodiesterase inhibitors show novel binding of boron to PDE4 bimetal center. FEBS letters, 586(19), 3410-3414.
Galkin, A., Kulakova, L., Lim, K., Chen, C. Z., Zheng, W., Turko, I. V., & Herzberg, O. (2014). Structural basis for inactivation of Giardia lamblia carbamate kinase by disulfiram. J Biol Chem, 289(15), 10502-10509. https://doi.org/10.1074/jbc.M114.553123
Grocin, A. G., Kallemeijn, W. W., & Tate, E. W. (2021). Targeting methionine aminopeptidase 2 in cancer, obesity, and autoimmunity. Trends Pharmacol Sci, 42(10), 870-882.
Han, B., & Weiss, L. M. (2018). Therapeutic targets for the treatment of microsporidiosis in humans. Expert Opinion on Therapeutic Targets, 22(11), 903-915. https://doi.org/10.1080/14728222.2018.1538360
Hughes, T. E., Kim, D. D., Marjason, J., Proietto, J., Whitehead, J. P., & Vath, J. E. (2013). Ascending dose-controlled trial of beloranib, a novel obesity treatment for safety, tolerability, and weight loss in obese women [Multicenter Study

### Randomized Controlled Trial

Research Support, Non-U.S. Gov't]. Obesity, 21(9), 1782-1788. https://doi.org/10.1002/oby.20356
Ingber, D., Fujita, T., Kishimoto, S., Sudo, K., Kanamaru, T., Brem, H., & Folkman, J. (1990). Synthetic analogues of fumagillin that inhibit angiogenesis and suppress tumour growth [In Vitro

### Research Support, Non-U.S. Gov't

Research Support, U.S. Gov't, P.H.S.]. Nature, 348(6301), 555-557. https://doi.org/10.1038/348555a0
Jacobs, R. T., Nare, B., Wring, S. A., Orr, M. D., Chen, D., Sligar, J. M., . . . Don, R. (2011). SCYX-7158, an orally-active benzoxaborole for the treatment of stage 2 human African trypanosomiasis. PLoS Negl Trop Dis, 5(6), e1151. https://doi.org/10.1371/journal.pntd.0001151
Killough, J. H., Magill, G. B., & Smith, R. C. (1952). The treatment of amebiasis with fumagillin. Science, 115(2977), 71-72.
Kulakova, L., Galkin, A., Chen, C. Z., Southall, N., Marugan, J. J., Zheng, W., & Herzberg, O. (2014). Discovery of novel antigiardiasis drug candidates. Antimicrobial agents and chemotherapy, 58(12), 7303-7311.
Kusaka, M., Sudo, K., Fujita, T., Marui, S., Itoh, F., Ingber, D., & Folkman, J. (1991). Potent anti-angiogenic action of AGM-1470: comparison to the fumagillin parent. Biochemical and Biophysical Research Communications, 174(3), 1070-1076. http://ww.ncbi.nlm.nih.gov/pubmed/1705118
Lanternier, F., Boutboul, D., Menotti, J., Chandesris, M. O., Sarfati, C., Mamzer Bruneel, M. F., . . . Lortholary, O. (2009). Microsporidiosis in solid organ transplant recipients: two Enterocytozoon bieneusi cases and review. Transpl Infect Dis, 11(1), 83-88. https://doi.org/10.111/i.1399-3062.2008.00347.x
Laupland, K. B., & Church, D. L. (2005). Population-based laboratory surveillance for Giardia sp. and Cryptosporidium sp. infections in a large Canadian health region. BMC Infect Dis, 5, 72. https://doi.org/10.1186/1471-2334-5-72
Lazarus, D., Doyle, E., Bernier, S., Rogers, A., Labenski, M., Wakefield, J., . . . Hoyt, J. (2008). An inhibitor of methionine aminopeptidase type-2, PPI-2458, ameliorates the pathophysiological disease processes of rheumatoid arthritis. Inflammation research, 57(1), 18-27.
Markinson, B., Ghannoum, M., Winter, T., Rycerz, A., Rock, F., & Gupta, A. K. (2018). Examining the Benefits of the Boron-Based Mechanism of Action and Physicochemical Properties of Tavaborole in the Treatment of Onychomycosis. J Am Podiatr Med Assoc, 108(1), 12-19. https://doi.org/10.7547/16-154
Martinez, M. N., & Amidon, G. L. (2002). A mechanistic approach to understanding the factors affecting drug absorption: a review of fundamentals. The Journal of Clinical Pharmacology, 42(6), 620-643.
Molina, J.-M., Goguel, J., Sarfati, C., Michiels, J.-F., Desportes-Livage, I., Balkan, S., . . . Struxiano, A. (2000). Trial of oral fumagillin for the treatment of intestinal microsporidiosis in patients with HIV infection. Aids, 14(10), 1341-1348.
Molina, J. M., Tourneur, M., Sarfati, C., Chevret, S., de Gouvello, A., Gobert, J. G., . . . Derouin, F. (2002).

Fumagillin treatment of intestinal microsporidiosis [Clinical Trial

### Randomized Controlled Trial

Research Support, Non-U.S. Gov't]. The New England journal of medicine, 346(25), 1963-1969. https://doi.org/10.1056/NEJMoa012924
Nazarian, R., & Weinberg, J. M. (2009). AN-2728, a PDE4 inhibitor for the potential topical treatment of psoriasis and atopic dermatitis. Curr Opin Investig Drugs, 10(11), 1236-1242.
Nocentini, A., Supuran, C. T., & Winum, J.-Y. (2018). Benzoxaborole compounds for therapeutic uses: a patent review (2010-2018). Expert opinion on therapeutic patents, 28(6), 493-504.
Osnat Herzberg, L. K., Janak Padia. (2019). Compounds useful for the treatment of giardiasis and amoebiasis. U.S. Ser. No. 16/551,628.
Padia, J., Kulakova, L., Galkin, A., & Herzberg, O. (2020). Discovery and preclinical development of antigiardiasis fumagillol derivatives. Antimicrobial agents and chemotherapy, 64(10), e00582-00520.
Pottorf, T. S., Fagan, M. P., Burkey, B. F., Cho, D. J., Vath, J. E., & Tran, P. V. (2020). MetAP2 inhibition reduces food intake and body weight in a ciliopathy mouse model of obesity. JCI Insight, 5(2). https://doi.org/10.1172/jci.insight.134278
Priest, R., Spaull, J., Buckton, J., Grimley, R., Sims, M., Binks, M., & Malhotra, R. (2009).

Immunomodulatory activity of a methionine aminopeptidase-2 inhibitor on B cell differentiation. Clinical & Experimental Immunology, 155(3), 514-522.
Richardson, P. G., Hideshima, T., & Anderson, K. C. (2003). Bortezomib (PS-341): a novel, first-in-class proteasome inhibitor for the treatment of multiple myeloma and other cancers. Cancer control, 10(5), 361-369.
Sin, N., Meng, L., Wang, M. Q., Wen, J. J., Bornmann, W. G., & Crews, C. M. (1997). The anti-angiogenic agent fumagillin covalently binds and inhibits the methionine aminopeptidase, MetAP-2. Proc Natl Acad Sci USA, 94(12), 6099-6103. https://doi.org/10.1073/pnas.94.12.6099
Sundqvist, M., Lundahl, A., Nagard, M., Bredberg, U., & Gennemark, P. (2015). Quantifying and communicating uncertainty in preclinical human dose-prediction. CPT: pharmacometrics & systems pharmacology, 4(4), 243-254.
Wentworth, J. M., & Colman, P. G. (2020). The methionine aminopeptidase 2 inhibitor ZGN-1061 improves glucose control and weight in overweight and obese individuals with type 2 diabetes: A randomized, placebo-controlled trial. Diabetes Obes Metab, 22(7), 1215-1219. https://doi.org/10.1111/dom.14009
Yanase, T., Tamura, M., Fujita, K., Kodama, S., & Tanaka, K. (1993). Inhibitory effect of angiogenesis inhibitor TNP-470 on tumor growth and metastasis of human cell lines in vitro and in vivo. Cancer Res, 53(11), 2566-2570.
Yang, F., Zhu, M., Zhang, J., & Zhou, H. (2018). Synthesis of biologically active boron-containing compounds. MedChemComm, 9(2), 201-211.
Zampino, M., Ferrucci, L., Spencer, R., Fishbein, K., Simonsick, E., & Kuo, P.-L. (2020). Poor Mitochondrial Health and Systemic Inflammation?Testing of a Classical Hypothesis. Innovation in Aging, 4(Supplement_1), 126-127.
Zheng, W., Chen, C., Marugan, J. J., Southall, N., Austin, C. P., Galkin, A., . . . Herzberg, O. (2015). Methods of treating giardiasis. In: Patents-U.S. Pat. No. 9,173,898B2.

1. A compound having the structure of Formula I, including pharmaceutically acceptable prodrugs, metabolites, and isomers thereof 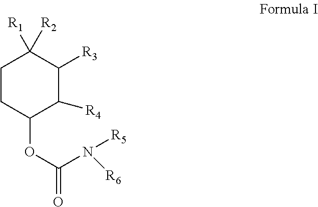
wherein
R₁ is selected from a group of —CH₂Cl, and —CH₂Br;
R₂ is selected from a group of hydrogen and hydroxyl;
R₁ and R₂ together can form 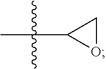 [SMILES: *C1CO1]
R₃ is selected from a group of hydrogen and substituted or unsubstituted alkyl;
R₄ is selected from a group of hydrogen and alkoxy;
R₅ is selected from a group of hydrogen and alkyl; and
R₆ is selected from a group of substituted or unsubstituted alkyl boronic acids, substituted or unsubstituted alkyl boronic acid esters, substituted or unsubstituted aryl boronic acids, substituted or unsubstituted aryl boronic acid esters, substituted or unsubstituted heterocyclic boronic acids, substituted or unsubstituted heterocyclic boronic acid esters;
NR₅R₆ can form a substituted or unsubstituted heterocyclic boronic acids, substituted or unsubstituted heterocyclic boronic acid esters.

2. The compound of claim 1, wherein R₁ and R₂ together can form

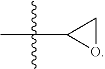 [SMILES: *C1CO1]

3. The compound of claim 1 wherein R₃ is selected from a group of

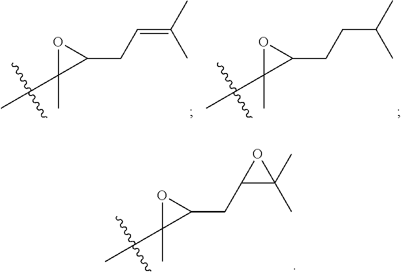

4. The compound of claim 1, wherein R₄ is methoxy.

5. The compound of claim 1, wherein R₅ is selected from a group of hydrogen, methyl, and ethyl.

6. The compound of claim 1, wherein R₆ is selected from a group of substituted or unsubstituted alkyl boronic acids, substituted or unsubstituted alkyl boronic acid esters, substituted or unsubstituted aryl boronic acids, substituted or unsubstituted aryl boronic acid esters, substituted or unsubstituted heterocyclic boronic acids, and substituted or unsubstituted heterocyclic boronic acid esters.

7. The compound of claim 1, wherein compound is selected having Formula II 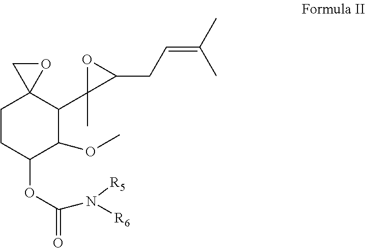
wherein
R₅ is selected from a group of hydrogen and alkyl; and
R₆ is selected from a group consisting of: 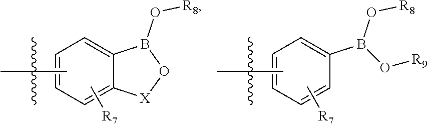
Wherein
R₇ is one or more substituent selected from a group of hydrogen, amino acid, halogen, alkylthio, alkyl, alkenyl, alkynyl, heteroalkyl, aryl, arylalkyl, heteroaryl, heteroarylalkyl, cycloalkyl and heterocycloalkyl wherein said amino acid, alkyl, alkenyl, alkynyl, heteroalkyl, aryl, arylalkyl, heteroaryl, heteroarylalkyl, cycloalkyl and heterocycloalkyl each is optionally substituted;
R₈ and R₉ are independently selected from a group of hydrogen, substituted or unsubstituted alkyl and
R₈ and R₉ can form a moiety derived from a dihydroxy compound having at least two hydroxy groups separated by at least two connecting atoms in a chain or ring, said chain or ring comprising carbon atoms, and optionally, a heteroatom or heteroatoms which can be N. S. or O; and
X is (CR₁₀R₁₁)ₙ wherein R₁₀ and R₁₁ are independently selected from hydrogen and alkyl, and
n=1 and 2.

8. The compound of claim 1 wherein compound is selected having Formula III 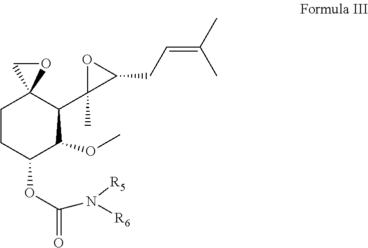
wherein
R₅ and R₆ are as defined in claim 1.

9. The compound of claim 1 wherein compound is selected from Formula IV 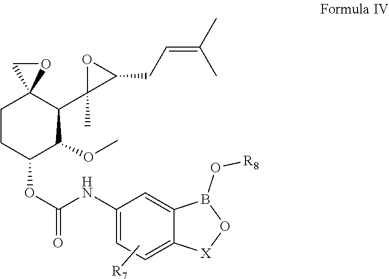
Wherein
R₇ and R₈ are as defined in claim 7.

10. The compound of claim 1 wherein compound is selected from Formula V 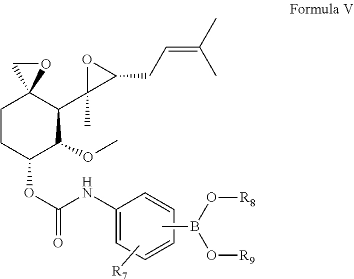
Wherein
R₇, R₈ and R₉ are as defined in claim 7.

11. The compound of claim 1 wherein compound is selected having Formula VI 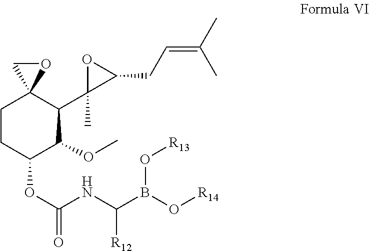
Wherein
R₁₂ is selected from a group of hydrogen, methyl, ethyl, propyl, isopropyl, butyl, isobutyl, pheny, benzyl, and
R₁₃ and R₁₄ are independently selected from a group of hydrogen, substituted or unsubstituted alkyl and
R₁₃ and R₁₄ can form a moiety derived from a dihydroxy compound having at least two hydroxy groups separated by at least two connecting atoms in a chain or ring, said chain or ring comprising carbon atoms, and optionally, a heteroatom or heteroatoms which can be N. S. or O.

12. The compound of claim 1 selected from Table A.

13. A method of use of a compound of Formula I as defined in the claim 1 for therapeutic use in a patient.

14. A method of use of according to claim 13 using a pharmaceutical composition for treating an immune reaction that results in pathology in a patient, comprising: a therapeutically effective amount of a compound selected from compounds of claim 1, said compound being capable of altering an aspect of MetAP2 metabolism or MetAP2 structure in a patient so as to result in treatment of said immune reaction.

15. A method of use of according to claim 13 using a pharmaceutical composition for treating an immune reaction that results in pathology in a patient, comprising: a therapeutically effective amount of a compound selected from compounds of claim 1, said compound being capable of treating and preventing cancer.

16. A method of use of according to claim 13 for treating intestinal parasitic diseases, comprising administering to a subject in need thereof a therapeutically effective amount of a compound of claim 1.

17. A method of use of according to claim 13, wherein the intestinal parasitic diseases are chosen from giardiasis, amebiasis, and a combination thereof.

18. A method of use of according to claim 13 for treating and/or controlling obesity, compromising administrating to a patient in need thereof an effective amount of a compound of claim 1.

19. A method of use of according to claim 13 for inducing weight loss in a patient in need thereof compromising administrating to a patient in need thereof an effective amount of a compound of claim 1.

20. A method of use of according to claim 13 for substantially preventing weight gain in a patient in need thereof an effective amount of a compound of claim 1.

21. The method of claim 13, wherein the compound is administered in or with one or more pharmaceutically acceptable carriers.

22. The method of claim 13, wherein the patient is human

23. The method of claim 13, wherein the patient is a cat or dog.

24. A kit comprising a compound of Formula I including pharmaceutically acceptable prodrugs, metabolites, and isomers thereof 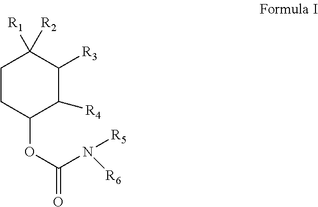
wherein
R₁ is selected from a group of —CH₂Cl, —CH₂Br, —CH₂1, —CH₂OSO₂CH₃, and —CH₂OCOCH₃;
R₂ is selected from a group of hydrogen and hydroxyl;
R₁ and R₂ together can form 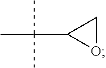
R₃ is selected from a group of hydrogen and substituted or unsubstituted alkyl;
R₄ is selected from a group of hydrogen and alkoxy;
R₅ is selected from a group of hydrogen and alkyl; and
R₆ is selected from a group of substituted or unsubstituted alkyl boronic acids, substituted or unsubstituted alkyl boronic acid esters, substituted or unsubstituted aryl boronic acids, substituted or unsubstituted aryl boronic acid esters, substituted or unsubstituted heterocyclic boronic acids, substituted or unsubstituted heterocyclic boronic acid esters.

25. The kit of claim 24, wherein the compound and the pharmaceutically acceptable carrier are in separate containers.

26. A process to produce a compound of claim 1 as described in schemes A-K.

27. The process of claim 26 to produce compounds of Table A.

28. The process of claim 27 to produce compound 1 (Table A) as described in scheme K and examples 1-8.
